(19) United States
(12) Patent Application Publication
(10) Pub. No.: US 2007/0010715 A1
(43) Pub. Date: Jan. 11, 2007

(54) Tissue retractor and method for using the retractor

(75) Inventors: Robert Sixto JR., Miami, FL (US); Juergen Kortenbach, Miami Springs, FL (US); Chris Martin, Miami, FL (US); Jorge Pinos, Hialeah, FL (US)

Correspondence Address: GREGORY L. MAYBACK, P.A., 5722 S. FLAMINGO ROAD #232, FORT LAUDERDALE, FL, 33330, US

(73) Assignee: ID, LLC

(21) Appl. No.: 11/321,684

(22) Filed: Dec. 29, 2005

Related U.S. Application Data
(63) Continuation-in-part of application No. 10/252,079, filed on Sep. 20, 2002, now Pat. No. 7,033,378.
(63) Continuation-in-part of application No. 10/252,069, filed on Sep. 20, 2002, now Pat. No. 6,966,919.
(63) Continuation-in-part of application No. 10/252,078, filed on Sep. 20, 2002.
(63) Continuation-in-part of application No. 10/728,389, filed on Dec. 5, 2003.
(63) Continuation-in-part of application No. 10/728,385, filed on Dec. 5, 2003.
(60) Provisional application No. 60/640,684, filed on Dec. 30, 2004.

(51) Int. Cl. A61B 1/32

(52) U.S. Cl. 600/217

(57) ABSTRACT

A retractor for manipulating an object includes a body having proximal and distal ends, a retraction device, and an actuation device. The retraction device has a head connected to the distal end of the body, substantially rigid needles, and an actuator. The head has a pivot. The needles are pivotally connected to the head about the pivot. The actuator is operatively connected to the needles and is movably disposed within the head and/or the body. The actuation device is connected to the proximal end of the body. The actuation device is operatively connected to the actuator through the body and, upon actuation thereof, moves the actuator to selectively rotate the needles between a stowed position where the needles are stowed within the head and a fully extended position where the needles are extended out of the head.

### CROSS-REFERENCE TO RELATED APPLICATION

This application claims the benefit under 35 U.S.C. § 119(e) of U.S. provisional applications Nos. 60/431,083, 60/505,009, 60/505,010, 60/640,684, filed Dec. 5, 2002, Sep. 22, 2003, and Dec. 30, 2004, and is a continuation in part of copending U.S. patent application Ser. No. 10/252,079, filed Sep. 20, 2002 and entitled “Surgical Fastener Particularly for the Treatment of Gastroesophageal Reflux Disease (GERD),” is a continuation in part of copending U.S. patent application Ser. No. 10/252,069, filed Sep. 20, 2002, and entitled “Instrument for Applying a Surgical Fastener Particularly for the Transoral Treatment of Gastroesophageal Reflux Disease (GERD),” is a continuation in part of copending U.S. patent application Ser. No. 10/252,078, filed Sep. 20, 2002, and entitled “Method for the Surgical Application of a Fastener and the Endoluminal Treatment of Gastroesophageal Reflux Disease (GERD),” is a continuation in part of copending U.S. patent application Ser. No. 10/728,389, filed Dec. 5, 2003, and entitled “Tissue Retractor and Method for Using the Retractor”, and is a continuation in part of copending U.S. patent application Ser. No. 10/728,385, filed Dec. 5, 2003, and entitled “Tissue Retractor and Method for Using the Retractor.” The entire disclosure of each of these applications is hereby incorporated herein by reference in their entireties.

### BACKGROUND OF THE INVENTION

1. Field of the Invention

The present invention relates to a tissue retractor, especially a flexible tissue retractor used as an endoscopic device that is passed through a working channel of a flexible endoscope. The tissue retractor has application in endoscopic and open surgery, including flexible endoscopy, laparoscopy, and general surgery. It can be made rigid or flexible and in lengths and diameters to suit the requirements of the surgical field. The flexible endoscopic tissue retractor is used, for example, to hold gastrointestinal tissue so that it can be retracted or manipulated in some way. The tissue retractor can be configured to allow grasping of specific layers of the gastrointestinal wall by adjusting the shape and/or length of the needles and their exit points at the tip of the device. For example, it can be configured to grasp through the mucosal layer, and into the muscular layer, thus providing a more secure connection to the tissue and allowing manipulation of the entire thickness of the tissue. Alternately, it can be configured to grasp the mucosal layer allowing manipulation of the mucosal layer only.

2. Description of Related Prior Art

A number of conventional devices exist in the prior art, which devices are used to manipulate the tissue during the endoscopic surgical procedure for treatment of Gastroesophageal Reflux Disease (GERD).

For example, U.S. Pat. No. 6,494,888 B1 to Laufer et al. (referred to hereinafter as “Laufer”) describes an instrument for reconfiguring stomach tissue. A tissue manipulator 700 includes an elongated cable assembly 716 and a distal end effector 718 actuated by the cable assembly 716 to perform various steps in the tissue reconfiguring procedure. See Laufer at FIGS. 9A to 9F. The end effector 718 has two jaw members 720, 722 that engage tissue, in particular, tissue at the gastroesophageal junction (GEJ). During the process of implanting the two-part fastener 732, 734 (see Laufer at FIG. 8), a coil 740 is rotated into the GEJ tissue and, after being screwed therein to a sufficient extent, is used to pull the GEJ tissue between the opening defined by the two jaw members 720, 722 in an open position illustrated, for example, in FIGS. 9D and 9E. The coil tissue puller 740, 741, 742 is shown, in particular, in FIG. 3D. The puller has certain disadvantages, however. The coil 740 can penetrate too far, causing possible negative consequences if the stomach is entirely breached (through the mucosa, muscularis, and serosa layers). Because the aorta, liver, diaphragm and other vital organs are disposed adjacent to the fundus of the stomach, if the coil 740 passes through the serosa, there is a significant chance of damage to the vital organs. Also, upon withdrawal, the coil 740, due to its inherent shape, can become stuck in the tissue and, thereby, cause damage to the tissue when the user must forcefully retract the entire assembly 718. Depending on the angle of entry, it is possible that the coil 740 only enters the mucosa. If this occurs, because the mucosa is a relatively thin, loosely attached layer, there is a high probability that the fastener 732, 734 will be only implanted in the mucosa and, therefore, result in a failed implantation procedure. Also, for fasteners that coil into the tissue, the tissue is compressed disadvantageously because rotation of the coil can twist the tissue as the coil is threaded in, which twisting can damage the tissue and cause it to weaken. Also, to advance the coil into the tissue, the coil must be rotated. It is inherently more difficult to transmit torque through a slender flexible device than it is to transmit thrust loads, thus, pushing the needles into the tissue is a more reliable actuation measure than twisting the coil into the tissue. Also, because the forces applied to the tissue by the engaging point of the device is not accompanied by an opposite reacting force of another engaging point of the device, all reaction forces must be provided through the shaft of the device.

A common general flexible endoscopic tissue grasper is most widely used today for manipulating gastrointestinal tissue (for example, one that is made by the Olympus company under the name Olympus Grasping Forceps (Catalog Number FG-49L-1)). A drawback to the Olympus grasper is its inability to reliably grasp muscularis through the mucosal layer. Another drawback is the requirement to maintain pressure on the handle while grasping the tissue. This ties up the user's hands and could lead to inadvertent release of the tissue.

The prior art devices are not constructed to easily, securely, selectively, and precisely engage the tissue during the surgical procedure.

### SUMMARY OF THE INVENTION

As it is well known, the tissue in the alimentary tract has three main layers that are, from the innermost layer to outermost layer, the mucosa, the muscularis, and the serosa. The mucosa is a relatively thin layer, loosely attached to the muscularis, and retraction of the mucosa only will not provide a sufficient plication for insertion of a fastener for the treatment of GERD. Retraction of entire thickness of the stomach wall is desired, as such retraction will provide a beneficial plication for insertion of a GERD-treating plication fastener. It is not desirable to perforate the serosal layer without having a fluid-tight seal. One of the most significant reasons is that an unsealed perforation of the serosa, if sufficiently large, could allow leakage of gastric contents into the peritoneal or thoracic cavities causing a potentially fatal infection.

It is accordingly an object of the present invention to provide a tissue retractor and method for using the retractor that overcome the hereinafore-mentioned disadvantages of the heretofore-known devices and methods of this general type and that can effectively grab the tissue, for example, of the alimentary tract during operation and avoid reaching into the serosa, and that can grab the tissue without compressing and/or tearing the tissue.

Various endoscopic procedures require manipulation of specific layers in the gastric wall. For instance, in the case of mucosal resection, the mucosa is tented away from the muscularis and resected away. Such a procedure is currently performed by injecting fluid beneath the mucosa to, thus, lift the mucosa from the muscularis. The mucosal tissue is, then, resected using electrocautery. The tissue retractor of the present invention can be used to selectively grasp the mucosa and lift it from the muscularis, thus enabling and simplifying mucosal resection. In the case of forming a full thickness plication in the stomach, the stronger muscular layer of the gastric wall must be grasped to ensure that the full thickness of the wall will be retracted when forming the plication. By tailoring the needles and the way in which they exit from the tip of the retractor, the retractor can be made to selectively grasp the different layers in the gastric wall. Being able to grasp a specific layer of the gastrointestinal wall is advantageous depending on the requirements of the specific procedure being performed.

The tissue retractor of the present invention has applications in laparoscopic and general surgery as well. It can be used to retract organs that are in the way of the surgical field, or to appose and hold tissue in place during suturing. An advantage to an organ retractor or tissue apposition device according to the present invention is the ability to retain the tissue without having to clamp onto it. The tissue retractor atraumatically retains the tissue by penetrating it with fine needles. To further reduce the trauma to the tissue, the needles can be formed with a conical point instead of a faceted point. This is especially advantageous when retracting sensitive organs such as the pancreas. Currently available tissue graspers use more aggressive serrated articulating end effectors, which require clamping forces to retain the tissue and, therefore, potentially cause trauma in the process.

A common procedure during flexible endoscopy is the exchange of an endoscope during a procedure. If the first scope is in a position within the alimentary tract that was difficult to achieve, and it is desired that the second (exchange) scope be in the same position, the tissue retractor could be used to guide the second scope into the position of the first scope. A flexible endoscopic version of the retractor according to the present invention can be provided with a removable handle. Therefore, when a scope exchange is necessary, the tissue retractor can be passed through the first scope and deployed in the tissue at the desired location. The handle of the tissue retractor can, then, be removed. The first scope can, then, be slid over the tissue retractor shaft, leaving the retractor shaft in-place, and removed. Then, the second scope can be fed over the tissue retractor shaft, much like a guidewire, and the scope advanced to the original position. Thereafter, the shaft can be released and removed when desired.

Also, a version of the retractor can be made that allows the distal tip of the retractor to be deployed in the tissue and, then, decoupled from the main shaft. In such an embodiment, the distal tip of the device is coupled removably to the shaft and the actuation wire is coupled removably to the needles. The needles are deployed on the target tissue and the shaft of the device is pulled proximally, thus allowing the actuation wire to slip free of the needles and the tip to slide free of the shaft. The released tip being firmly attached to the tissue has application as a marker, as suture attachment points for a purse string closure, as a tissue apposition suture, and as an anchoring point for various things such as pH probes, miniature capsule cameras, and feeding tubes.

The device and method of the present invention allows the needles to be configured such that they can be made to penetrate deep through the mucosa and into the muscularis, making a more secure attachment to the tissue, while substantially reducing the possibility of puncturing the serosa, or penetrate less deep to grasp only the mucosal layer. The present invention, in the two-needle embodiment, engages the tissue at two opposing points so that the tissue-engaging forces of each point react against the forces of the other; the result is that there is very little reaction load transmitted to the flexible shaft of the device. This deployment of the device does not require substantial torque or thrust loads to be supplied by the shaft. The present invention provides better visibility during placement of the retractor as no jaws are used that could obscure a view of the retraction site. It is also less traumatic to the tissue than a conventional articulating grasper due to the fine diameter and non-cutting points of the needles. The handle can be released from the user's grasp after the needles have been deployed, while still maintaining a secure attachment to the tissue, which frees the user to do other tasks after the tissue has been manipulated or is being manipulated. The tissue retractor is separate from an endoscope but sized to fit within a working channel of the endoscope.

A retractor for manipulating an object includes a body having proximal and distal ends, a retraction device, and an actuation device. The retraction device has a head connected to the distal end of the body, substantially rigid needles, and an actuator. The head has a pivot. The needles are pivotally connected to the head about the pivot. The actuator is operatively connected to the needles and is movably disposed within the head and/or the body. The actuation device is connected to the proximal end of the body. The actuation device is operatively connected to the actuator through the body and, upon actuation thereof, moves the actuator to selectively rotate the needles between a stowed position where the needles are stowed within the head and a fully extended position where the needles are extended out of the head.

Other features that are considered as characteristic for the invention are set forth in the appended claims.

Although the invention is illustrated and described herein as embodied in a flexible tissue retractor and method for using the retractor, it is, nevertheless, not intended to be limited to the details shown because various modifications and structural changes may be made therein without departing from the spirit of the invention and within the scope and range of equivalents of the claims.

The construction and method of operation of the invention, however, together with additional objects and advantages thereof, will be best understood from the following description of specific embodiments when read in connection with the accompanying drawings.

### BRIEF DESCRIPTION OF THE DRAWINGS

FIG. 1 is a fragmentary, perspective view from a side above a distal end of a first embodiment of a flexible tissue retractor according to the invention with needles in a deployed position;

FIG. 2 is a fragmentary, perspective side view of the distal end of the retractor of FIG. 1 with a cut-away portion of a left side of the outer jacket to show the interior of the retractor;

FIG. 3 is a fragmentary, perspective side view of the distal end of the retractor of FIG. 1 with a cut-away portion of a right side of the outer jacket to show the interior of the retractor;

FIG. 4 is a fragmentary, perspective side view of the distal end of the retractor of FIG. 3 with a greater cut-away portion of the right side of the outer jacket and with the needles in a slightly retracted position;

FIG. 5 is a fragmentary, perspective side view of the distal end of the retractor of FIG. 4 with the needles in a fully retracted position;

FIG. 6 is a fragmentary, side elevational view of the needles and the actuation rod of FIGS. 2 to 5;

FIG. 7 is a fragmentary, exploded view of the components of a larger portion of the retractor of FIG. 1;

FIG. 8 is a fragmentary, perspective and partially broken away view of the distal portion of the retractor of FIGS. 1 to 5 and 7;

FIG. 9 is a fragmentary, perspective view of a distal end of a flexible tissue retractor according to the invention with needles in a deployed position;

FIG. 10 is a fragmentary, side elevational and hidden view of a second embodiment of a flexible tissue retractor according to the invention with needles in a stowed position;

FIG. 11 is a fragmentary, side elevational and hidden view of the flexible tissue retractor of FIG. 10 with needles in a first partially deployed position;

FIG. 12 is a fragmentary, side elevational and hidden view of the flexible tissue retractor of FIG. 10 with needles in a second partially deployed position;

FIG. 13 is a fragmentary, side elevational and hidden view of the flexible tissue retractor of FIG. 10 with needles in a fully deployed position;

FIG. 14 is a fragmentary, perspective view from the bottom left of the flexible tissue retractor of FIG. 10 with needles in the fully deployed position;

FIG. 15 is a fragmentary, perspective view of the flexible tissue retractor of FIG. 10 rotated leftward approximately 60 degrees with needles in the stowed position;

FIG. 16 is a fragmentary, perspective view of the flexible tissue retractor of FIG. 10 rotated leftward approximately 45 degrees with needles in a partially deployed position;

FIG. 17 is a fragmentary, perspective view of the flexible tissue retractor of FIG. 16 with needles in a further partially deployed position;

FIG. 18 is a fragmentary, perspective view of the control assembly and the needles of the flexible tissue retractor of FIG. 17;

FIG. 19 is a fragmentary, perspective view of the flexible tissue retractor of FIG. 15 with needles in yet another partially deployed position;

FIG. 20 is a fragmentary, side elevational view of the flexible tissue retractor of FIG. 10;

FIG. 21 is a fragmentary, side elevational view of the flexible tissue retractor of FIG. 10 with the clevis assembly removed;

FIG. 22 is a fragmentary, side elevational view of the flexible tissue retractor of FIG. 20 with the needles in a partially deployed position;

FIG. 23 is a fragmentary, side elevational view of the flexible tissue retractor of FIG. 20 with the needles in a further partially deployed position;

FIG. 24 is a fragmentary, side elevational view of the flexible tissue retractor of FIG. 23 with the clevis assembly removed;

FIG. 25 is a fragmentary, side elevational view of the flexible tissue retractor of FIG. 20 with the needles in a fully deployed position;

FIG. 26 is a fragmentary, side elevational view of the flexible tissue retractor of FIG. 25 with the clevis assembly removed;

FIG. 27 is a fragmentary, side elevational view of a third embodiment of a distal portion of a flexible tissue retractor according to the invention with half of the nose assembly removed and with needles in a stowed position;

FIG. 28 is a fragmentary, side elevational view of the flexible tissue retractor of FIG. 27 with needles in a first partially deployed position;

FIG. 29 is a fragmentary, side elevational view of the flexible tissue retractor of FIG. 27 with needles in a second partially deployed position;

FIG. 30 is a fragmentary, side elevational view of the flexible tissue retractor of FIG. 27 with needles in a third partially deployed position;

FIG. 31 is a fragmentary, side elevational view of the flexible tissue retractor of FIG. 27 with needles in a fully deployed position;

FIG. 32 is a fragmentary, enlarged, side elevational view of the flexible tissue retractor of FIG. 27;

FIG. 33 is a fragmentary, enlarged, side elevational view of the flexible tissue retractor of FIG. 28;

FIG. 34 is a fragmentary, enlarged, side elevational view of the flexible tissue retractor of FIG. 29;

FIG. 35 is a fragmentary, enlarged, side elevational view of the flexible tissue retractor of FIG. 30;

FIG. 36 is a fragmentary, enlarged, side elevational view of the flexible tissue retractor of FIG. 31;

FIG. 37 is a fragmentary, perspective view of the distal portion of the flexible tissue retractor of FIG. 27 with needles in a fully deployed position;

FIG. 38 is a fragmentary, perspective view of the distal portion of the flexible tissue retractor of FIG. 37 with needles in a partially deployed position;

FIG. 39 is a fragmentary, perspective view of the distal portion of the flexible tissue retractor of FIG. 37 with needles in a fully stowed position;

FIG. 40 is a fragmentary, perspective view of the distal portion of the flexible tissue retractor of FIG. 27 with a protective flexible sheath surrounding an actuation assembly and needles in a fully deployed position;

FIG. 41 is a fragmentary, perspective view of the flexible tissue retractor of FIG. 40 with the flexible sheath removed;

FIG. 42 is a fragmentary, perspective and hidden view of the flexible tissue retractor of FIG. 41 rotated counterclockwise with respect to FIG. 41 and with hidden lines indicating components inside the distal clevis;

FIG. 43 is a fragmentary, perspective view of the flexible tissue retractor of FIG. 42 with half of the distal clevis removed;

FIG. 44 is a fragmentary, perspective view of the flexible tissue retractor of FIG. 41 with needles in a stowed position;

FIG. 45 is a fragmentary, perspective and hidden view of the flexible tissue retractor of FIG. 44 with hidden lines indicating components inside the distal clevis;

FIG. 46 is a fragmentary, perspective and hidden view of the flexible tissue retractor of FIG. 42 with the retractor rotated approximately 180 degrees;

FIG. 47 is a side elevational view of a fourth embodiment of a distal portion of a flexible tissue retractor according to the invention with needles in a stowed position;

FIG. 48 is a side elevational view of the flexible tissue retractor of FIG. 47 with needles in a first partially deployed position;

FIG. 49 is a side elevational view of the flexible tissue retractor of FIG. 47 with needles in a second partially deployed position;

FIG. 50 is a side elevational view of the flexible tissue retractor of FIG. 47 with needles in a third partially deployed position;

FIG. 51 is a side elevational view of the flexible tissue retractor of FIG. 47 with needles in a fully deployed position;

FIG. 52 is a side elevational view of the flexible tissue retractor of FIG. 51 rotated approximately 45 degrees with respect to FIG. 51;

FIG. 53 is a perspective view of the flexible tissue retractor of FIG. 52 rotated in two degrees of freedom with respect to FIG. 52;

FIG. 54 is a perspective view of the flexible tissue retractor of FIG. 53 rotated by approximately 180 degrees with respect to FIG. 53;

FIG. 55 is a perspective view of the flexible tissue retractor of FIG. 54 with the proximal end of the clevis rotated to the right with respect to FIG. 54 and with needles in a less deployed position than FIG. 54; and

FIG. 56 is a perspective view of the flexible tissue retractor of FIG. 53 with the distal end of the clevis rotated to the left with respect to FIG. 53 and with needles in a less deployed position than FIG. 55.

FIG. 57 is a perspective view of a handle at a proximal end of the retractor according to the invention in a retracted position;

FIG. 58 is a fragmentary, cross-sectional view of some components of the handle of FIG. 57 in a deployed position;

FIG. 59 is an exploded view of some of the components of the handle of FIG. 57;

FIG. 60 is a fragmentary, cross-sectional view of some components of the handle of FIG. 57 in the retracted position;

FIG. 61 is a fragmentary, cross-sectional view of an enlarged portion of the distal components of the handle of FIG. 60;

FIG. 62 is a cross-sectional view of a button assembly of the handle of FIG. 57 along section line 62-62;

FIGS. 63 to 70 are fragmentary, partially cross-sectional and partially plan views of the method of using the retractor according to the invention to retract tissue at different retraction angles;

FIG. 71 is a fragmentary, partially cross-sectional and partially plan view of the method of using the retractor according to the invention with needle exit windows more proximal to the tip than in FIGS. 63 to 70;

FIG. 72 is a fragmentary, side view of a plication device coupled to an endoscope during insertion of the two into the stomach;

FIG. 73 is a fragmentary, perspective side view of the plication device separated from the endoscope and shown with the jaws in an open position;

FIG. 74 is a fragmentary, perspective side view similar to FIG. 73, and additionally shows the retractor according to the invention advanced through the endoscope and engaging the target tissue at which a plication is desired to be made;

FIG. 75 is a fragmentary, perspective side view shown with the jaws of the plication device plicating the target tissue and a plication fastener in a locked configuration;

FIG. 76 is a fragmentary, perspective side view shown with the jaws of the plication device opened around the target tissue and the fastener plicating the target tissue;

FIG. 77 is a fragmentary, perspective side view shown with the jaws of the plication device in an open position and the fastener disposed therein; and

FIGS. 78 through 88 are fragmentary partially cross-sectional and partially elevational views illustrating the procedure according to the invention in which the retractor is advanced through a working channel of an endoscope into the stomach and operated under view of the endoscope.

### DETAILED DESCRIPTION OF THE PREFERRED EMBODIMENTS

While the specification concludes with claims defining the features of the invention that are regarded as novel, it is believed that the invention will be better understood from a consideration of the following description in conjunction with the drawing figures, in which like reference numerals are carried forward.

Before the present invention is disclosed and described, it is to be understood that the terminology used herein is for the purpose of describing particular embodiments only and is not intended to be limiting. It must be noted that, as used in the specification and the appended claims, the singular forms “a,” “an,” and “the” include plural references unless the context clearly dictates otherwise.

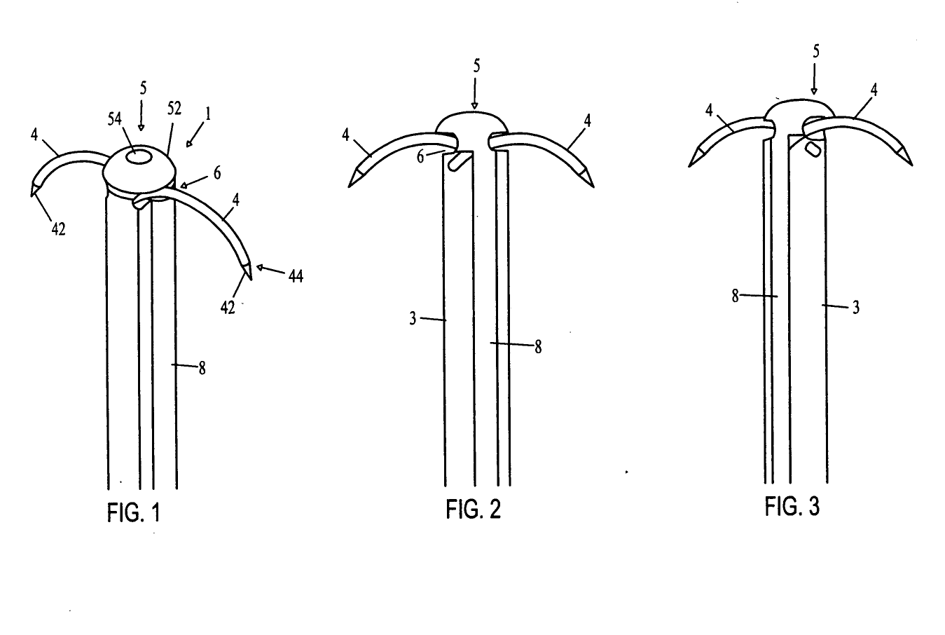

Referring now to the figures of the drawings in detail and first, particularly to FIG. 1 thereof, there is shown a perspective view of a distal portion of a flexible tissue retractor 1 according to the invention with needles 4 in a deployed or extended position.

A distal tip 5 is assembled at a distal end of an outer jacket 8. Preferably, the distal tip 5 is fastened to the distal end of the outer jacket 8. For example, the distal tip 5 can have a mushroom-shaped head 52 with a cylindrical body radially smaller than the head 52. The body can be slidably inserted into the hollow distal end of the outer jacket 8 and fastened thereto using any fastening measure. For example, the body can be welded, heat-shrunk, melted, or glued into the outer jacket 8 or the body can have a male thread that is screwed into a female thread disposed on the interior surface of distal end of the outer jacket 8. The distal tip 5 may be formed, for example, as a single piece or from two non-illustrated half-pieces secured together. If the distal tip 5 is constructed from two halves, then they can self-lock. Accordingly, the tip halves can each be formed with two holes and two pins, for example, with the holes and pins each respectively fitting into corresponding pins and holes formed in the other tip half to fix the two tip halves together. The tip halves need not be mirror-opposite. They can be identical with respect to the shape of the holes and pins. Of course, any similar fastening device, or combinations thereof, can be used to lock the tip halves to one another, i.e., screws, rivets, catch tabs and slots, and/or catch cylinders and holes.

The tip can be made from a thin walled deep drawn part with a rounded end to maximize the internal diameter of the tip, thus allowing for arcuate needles of greater chord height (shorter, smaller radius) to fit within. The exit windows for the needles can be pierced through the wall as part of the deep drawing operation or machined through using various methods including at least one of: wire EDM, laser, conventional milling, etc.

The distal tip 5 and the outer jacket 8 together define an exit window 6 at each side of the distal end for receiving therethrough a respective one of the needles 4. Alternatively, the distal tip 5 can solely define the exit window 6 at each side thereof for receiving therethrough a respective one of the needles 4. The needles 4 are, preferably, made of steel, and, therefore, substantially rigid, and are pre-formed into an arcuate shape. The needles 4 can also be made of ceramic, such as alumina or zirconia, or of a polymer, such as ULTEM® or a liquid crystal polymer.

FIGS. 2 to 5 are side views with various cut-away portions of the outer jacket 8 showing the components in the interior of the outer jacket 8.

In FIGS. 2 and 3, the needles 4 are fully extended. The cut-away view of FIG. 2 illustrates the preferred connection of a first side of the right-extending needle 4 (as viewed in FIG. 2) to a column-shaped actuation rod 3. The cut-away view of FIG. 3 illustrates the preferred connection of the first side of the left-extending needle 4 (as viewed in FIG. 3) to the actuation rod 3.

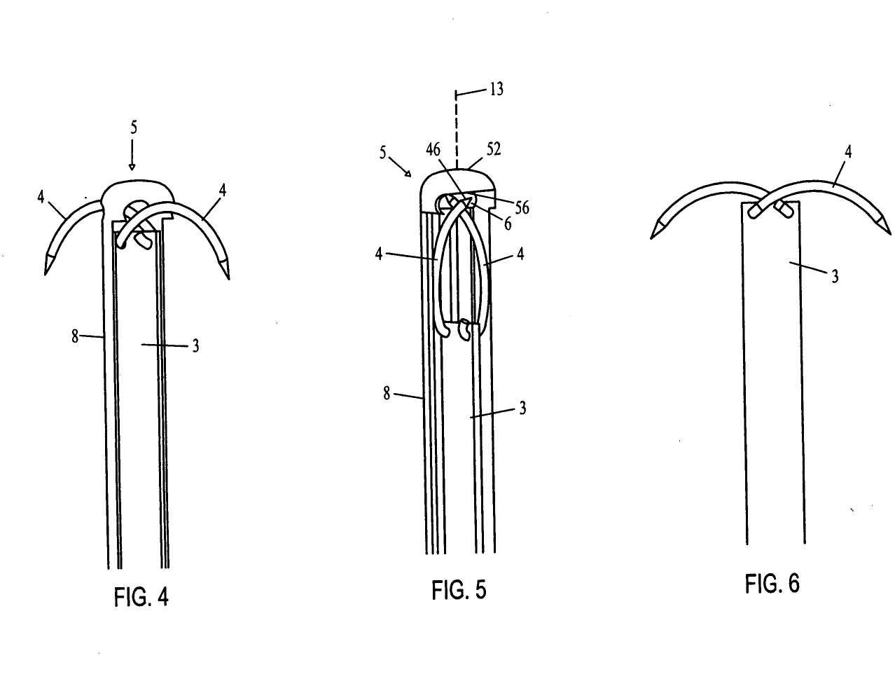

In FIG. 4, the needles 4 are partially retracted into the distal tip 5/outer jacket 8. The cut-away view of FIG. 4 illustrates the preferred connection of the needles 4 to the actuation rod 3 by showing connection of the first side of the right-extending needle 4 and of the second side of the left-extending needle 4 (as viewed in FIG. 4).

In FIG. 5, the needles 4 are fully retracted within the distal tip 5/outer jacket 8. As can be seen from FIGS. 2 to 5, as well as in FIG. 6 (in which the distal tip 5 and the outer jacket 8 are not shown for clarity), the needles 4 are connected movably to the actuation rod 3, preferably, pivotally to the rod 3. The needles 4 can be crimped, bent, or otherwise fastened to the actuation rod 3, or the needles 4 can be formed as an integral part of the actuation rod 3, so long as the needles 4 can move relative to the rod 3. The preferred embodiment of the retractor 1 has two needles 4. However, the number of needles can be reduced or expanded to suit particular needs.

FIGS. 1 to 5 show different views of the needles 4 between retracted and extended positions. The entirety of such movement is referred to as selective movement because actuation of the needles is selected by a user anywhere between the fully retracted and fully extended positions.

Due to the preferred rigid construction of the needles 4, they retain the arcuate shape throughout repeated retractions/deployments and are not intended to deform during the life of the retractor 1. Therefore, as used herein, the phrases “without substantial deformation” or “substantially non-deformable” or “substantially rigid” with respect to the needles 4 means that the needles 4 flex in the elastic range of the material with which the needles are made and can, in some circumstances, flex beyond the plastic range. These definitions do not include pseudo-elastic materials. When the needles 4 are made of steel, they flex in the elastic range typical of stainless steels such as, for example, hard-drawn 304 Steel and 17-7 condition C H900 steel and are not intended to be stressed beyond the respective yield point under normal use. The shape and location of the exit windows 6 are chosen so that the needles 4 can freely extend out of or retract into the distal tip 5/outer jacket 8 without substantial deformation. The needles 4 are curved so that each needle 4 is guaranteed to exit its respective window 6. It is noted that the device can also be made with a material that plastically deforms instead of only deforming elastically. In such a case, the device would not be used so many times that the needles would fail because of low-cycle fatigue. If, for example, fully-annealed Monel wire were used, the device could be cycled many times before failing even though it were deforming plastically.

In a preferred embodiment, therefore, when the needles 4 are fully retracted into the outer jacket 8 (as shown in FIG. 5), the needles 4 first curve away from the rod 3 so that a surface of the needles 4 that is radially furthest away from the distal tip axis 13 slightly contacts the interior surface of the distal tip 5/outer jacket 8 at a point 54 on a first longitudinal line (parallel to the axis 13) of the distal tip 5/outer jacket 8. Distal from the point 54, the needles 4 curve back towards the axis 13 and, preferably, past the axis 13 in a direction opposite (180°) the first longitudinal line. The curve back of the needles 4 can extend until the points 42 of the needles 4 almost contact the interior surface of the distal tip 5/outer jacket 8 near a point in a second longitudinal line (parallel to the axis 13) that is opposite (180°) the first longitudinal line. Alternatively, the needle points 42 can be configured to prevent snagging and, therefore, can extend to contact the interior surface of the distal tip 5/outer jacket 8.

The needles 4 have needle ends 44 that are, preferably, tapered. Therefore, as shown in the upper-most portion of FIG. 5, such a taper forms a cam follower 46 for the cam surface 56 on the inside/underside of the head 52 of the distal tip 5.

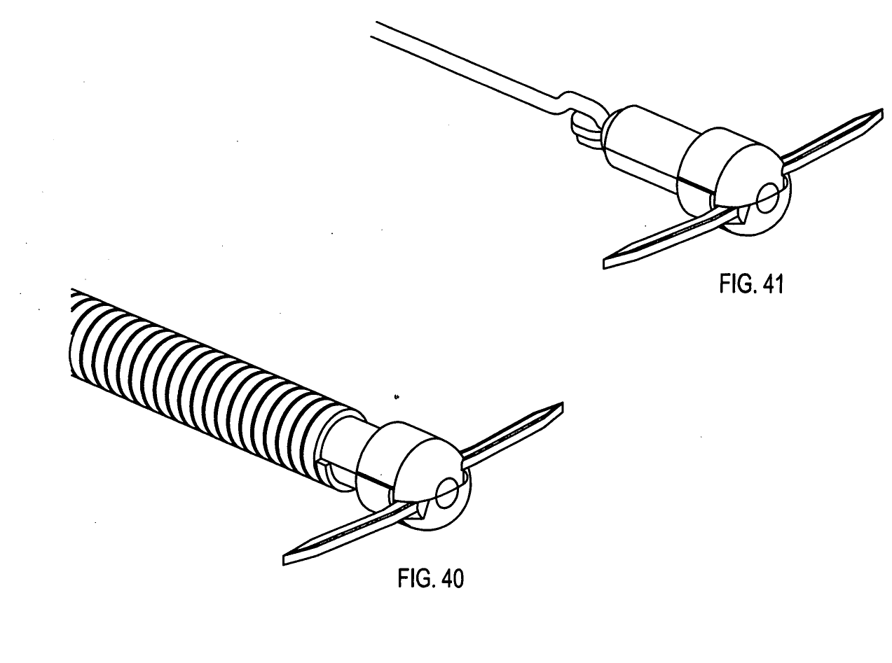

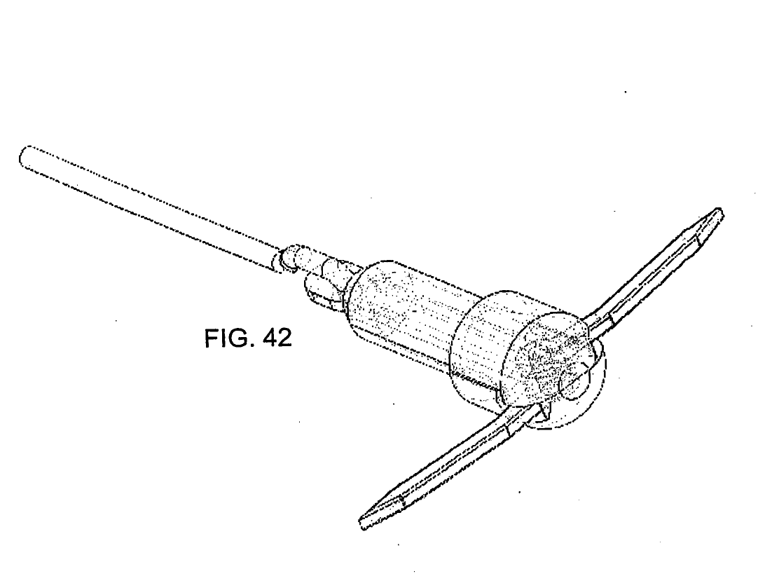

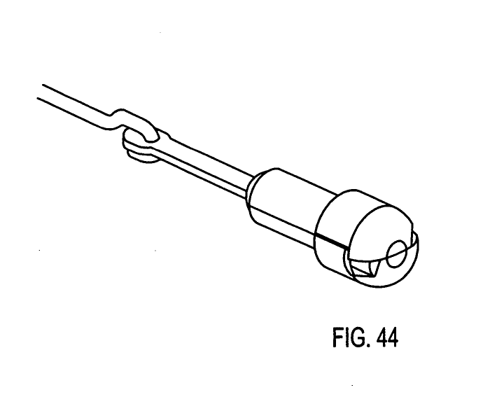

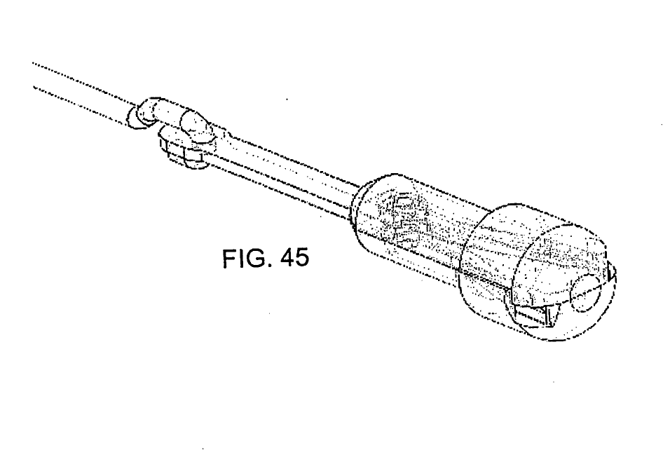

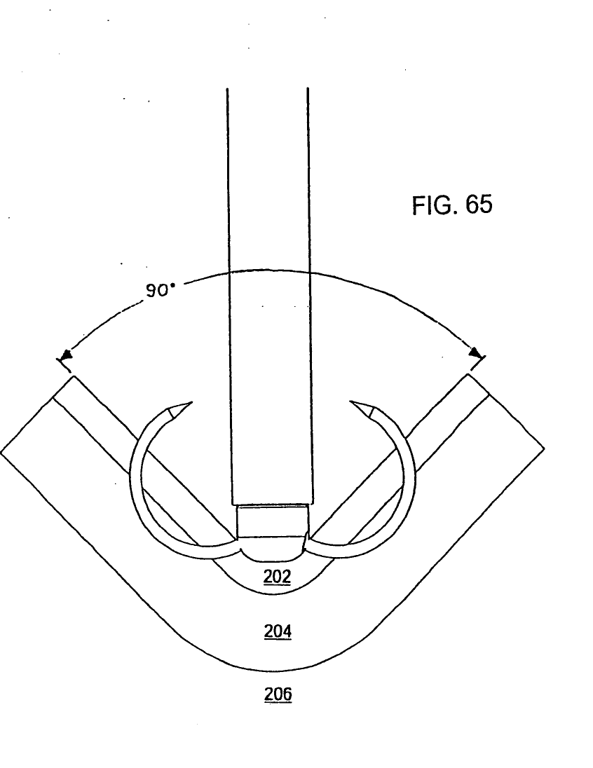

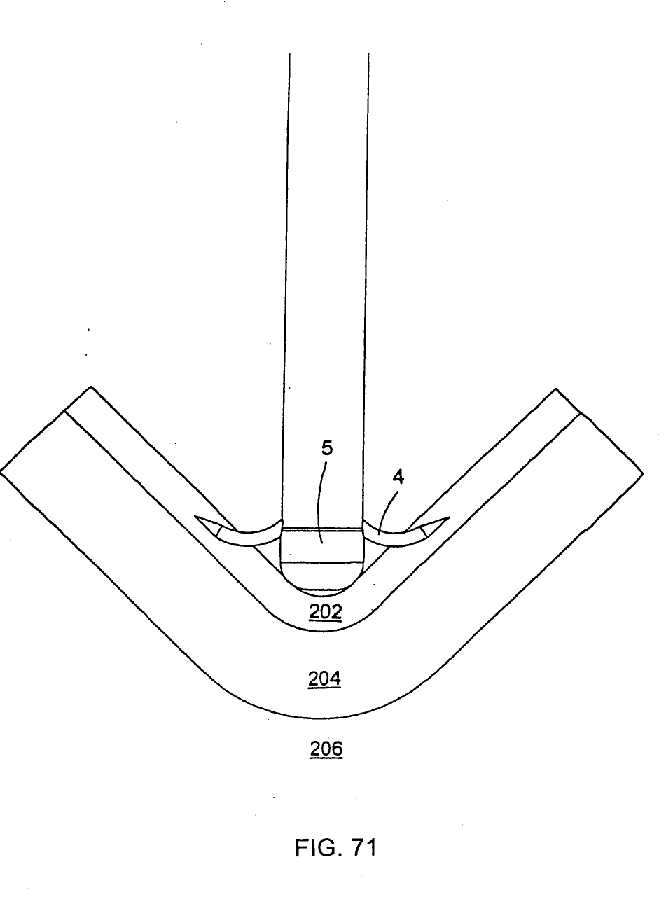

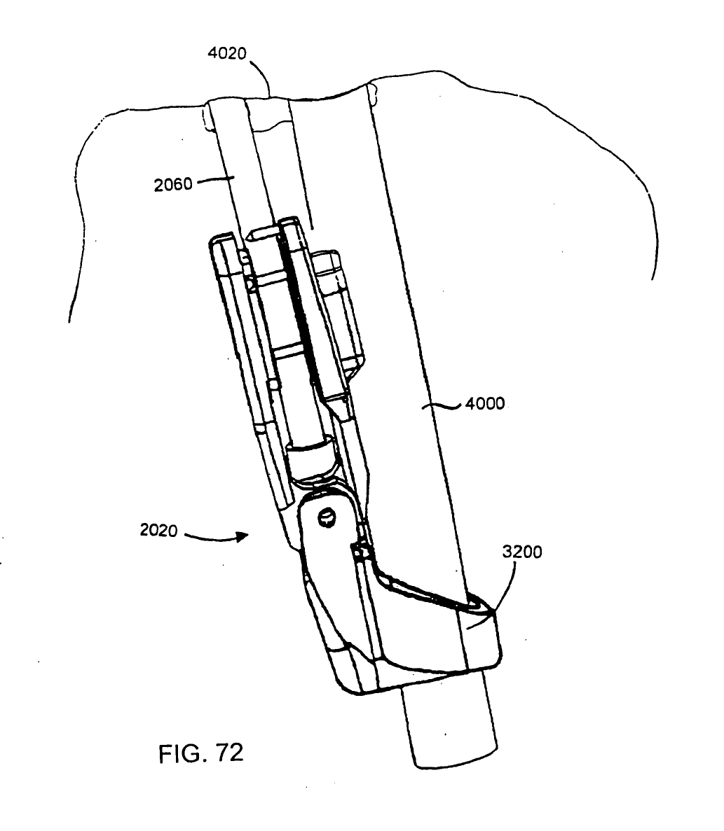

The retractor 1 can be configured to selectively grasp a desired number of layers depending upon the curve of the needles 4 and the size and/or orientation of the window 6. Particularly with regard to stomach tissue (see FIGS. 36 to 71), the retractor 1 can be used to selectively grasp the mucosa 202 and lift it from the muscularis 204, thus enabling and simplifying mucosal resection. In the case of forming a full thickness plication in the stomach, the stronger muscular layer of the gastric wall must be grasped to ensure that the full thickness of the wall will be retracted when forming the plication (see FIGS. 65 to 70, 75, 76, 85, and 86). By tailoring the needles 4 and the way in which they exit from the tip 5 of the retractor 1, the retractor 1 can be made to selectively grasp the different layers in the gastric wall. See, e.g., FIG. 71. Being able to grasp a specific layer of the gastrointestinal wall is advantageous depending on the requirements of the specific procedure being performed.

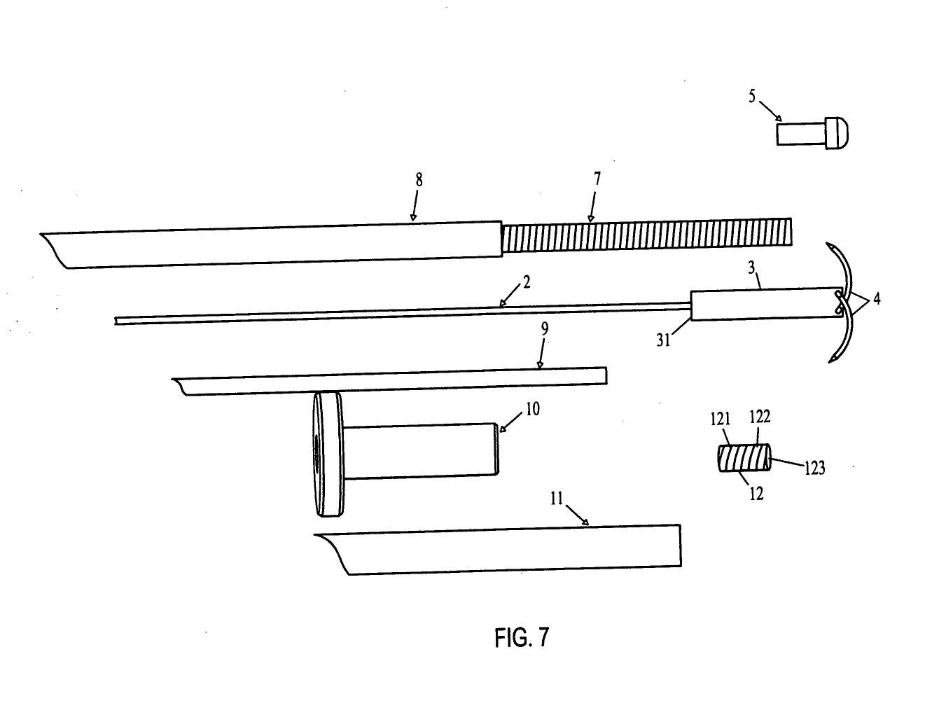

A larger portion of the retractor 1 is shown in the exploded view of FIG. 7, in which an outer jacket 8 surrounds a coil winding 7. Also shown are a sheath 9 (preferably, of polyethylene or TEFLON®), a coil connector 10, and a strain relief 11, each of which will be explained in further detail below.

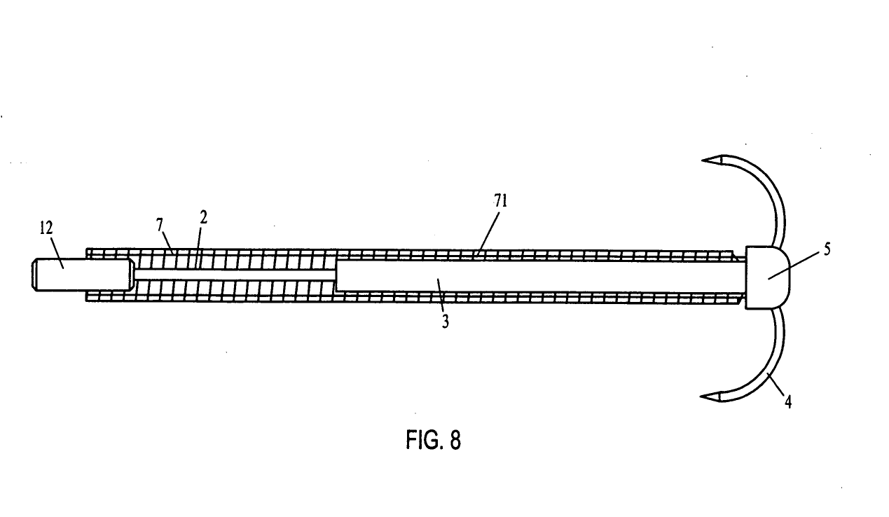

FIG. 8 illustrates the connection between the actuation wire 2 and the rod 3 in a partially broken away view of the distal end of the retractor 1. The rod 3 is, preferably, integral with the actuation wire 2. However, the rod 3 can be welded, bonded, or adhesively connected to the actuation wire 2. Alternatively, the rod 3 can have a non-illustrated proximal hollow crimp end that slidably receives the distal end of the actuation wire 2 therein and, thereafter, is squeezed by a mechanical stress to fixedly connect the actuation wire 2 to the rod 3. Alternatively, the proximal crimp end can be a heat-contacted sleeve in which heat welds, forms, molds, or otherwise shapes the body of the sleeve to affix the sleeve to the actuation wire 2.

In an alternative embodiment, it may be desirable to decouple the rod 3 from the actuation wire 2 selectively. In such an embodiment, after the distal tip 5 of the retractor 1 is deployed in the tissue, it can be decoupled from the main shaft (including the actuation wire 2, the coil winding 7, the outer jacket 8, and the sheath 9). To accomplish selective decoupling, the distal tip 5 is held loosely in at least one of the coil winding 7 and the outer jacket 8 and the actuation wire is coupled removably to the rod 3. For example, the actuation wire 2 can have a male threaded end screwed into a female threaded bore in the rod 3 and, after deploying the needles 4 in the tissue, the actuation wire 2 is unthreaded, thereby releasing the rod 3, with the needles 4 and tip 5, from the retractor 1. Other release devices can be used, such as a crimp of the rod 3 on the actuation wire 3 that is not permanent and can be overcome by a proximally directed force. When the released tip (3, 4, 5) is firmly attached to the tissue it can have application as a marker, suture attachment points for a purse string closure, a tissue apposition suture, and an anchoring point, for example, for various things such as pH probes, miniature capsule cameras, and feeding tubes.

The partially broken away view of FIG. 8 shows the actuation wire 2, the rod 3, and the needles 4, placed inside the coil winding 7, which is coaxially disposed within the outer jacket 8. A proximal stop 12 is located at the proximal end of the distal portion to limit the retraction range of the needles 4.

The coil winding 7 is, preferably, made of an ovular or circular wire that is wound, in the fashion of a tight spring, to provide it with longitudinal strength while having slight longitudinal expandability/give and to provide it simultaneously with radial flexibility or whip. Due to such coiling, the interior of the coil winding 7 has a natural female thread 71.

The stop 12 is hollow to accommodate the actuation wire 2 slidably therein (in a preferred embodiment, the sheath 9 is not allowed to pass through the stop 12). Thus, the internal diameter of the stop 12 is as least slightly greater than the external diameter of the actuation wire 2. The stop 12 is provided with a male thread 121 on its external surface. A groove 122 is provided at the distal end of the stop 12, the groove 122, preferably, being shaped to accommodate the working end of a flat-head screwdriver.

To load the assembly of FIG. 6 into the distal end of the outer jacket 8 and coil winding 7, the stop 12 is inserted into the interior of the coil winding 7 (by threading it therein using rotation of a screwdriver placed in the groove 122) to a given distance. The proximal end of the actuation wire 2 is threaded through the distal end of the coil winding 7 and through the hollow interior of the stop 12. Thus, when the actuation wire 2 is moved proximally, the proximal surface 31 of the rod 3 will, ultimately, contact the distal surface 123 of the stop 12 to prevent further proximal movement of the actuation wire 2 and halt retraction of the needles 4.

The actuation wire 2 is disposed to deploy and retract the needles 4. As can be seen from FIGS. 1 through 5, the needles 4 pass through the openings 6 at the distal tip 5 to extend out of the distal tip 5. FIGS. 4 and 6 are helpful particularly to explain the movement of the needles 4 through the distal tip 5. When the retractor 1 is in a position where tissue retraction is desired, the user moves the actuation wire 2 distally (as set forth in further detail below). As the needles 4 begin to move in a distal direction (in particular, linearly with respect to the longitudinal movement of the actuation wire 2), the tapered portion of each needle end 44 of the needles 4 contacts the interior/underside of the distal tip 5. Because the tapered portion forms an acute angle with respect to the interior/underside of the distal tip 5 (and because the needle points 42 do not catch upon the interior/underside of the distal tip 5), the interior/underside of the distal tip 5 acts as a cam surface 56 for the taper, which, because of its shape, become a cam follower 46. In such a configuration, the needles 4 are guaranteed to extend out of their respective windows 6 every time the needles 4 move in a distal direction. When the actuation wire 2 is moved proximally, the needles 4 withdraw into the distal tip 5 with the edge opening of the windows 6 each acting as a cam surface and a respective edge of each of the needles 4 bearing against the cam surface acting as a cam follower. As set forth above, the cam surface 56 can be varied, depending upon the procedure for using the retractor 1, so that the needles 4 extend deeper, shallower, more distal, less distal, more proximal, and/or less proximal.

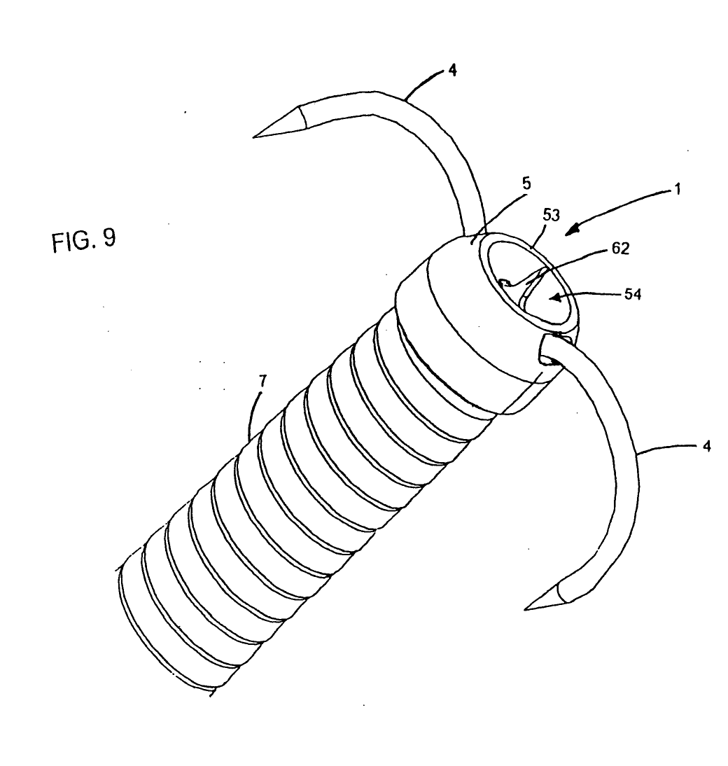

As shown in FIG. 9, a hollow or bowl 54 can be formed on the distal end face 53 of the distal tip 5 to accommodate therein tissue when the distal tip 5 is pressed against the tissue. An anchoring spike 62 may also be formed at the distal end face 53 of the distal tip 5, preferably, centered in the hollow 54. The function of the anchoring spike 62 is to keep in place and prevent the tip 5 from glancing off a tissue surface (i.e., human tissue, in particular, the wall of the stomach) when the tip 5 of the tissue retractor 1 is pushed initially against the tissue surface. It is noted that the hollow 54 allows the tissue surface to be compressed therein and around the spike 62 to secure the retractor 1 at a grasping location on the surface and prevent radial movement with respect to the spike 62. It is noted that this hollow 54 can be formed in each embodiment of the present invention and is not restricted to one embodiment.

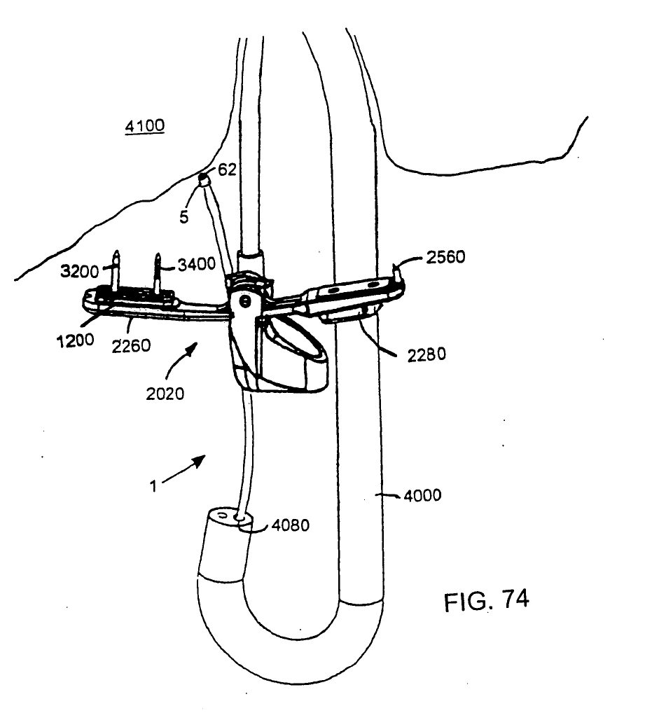

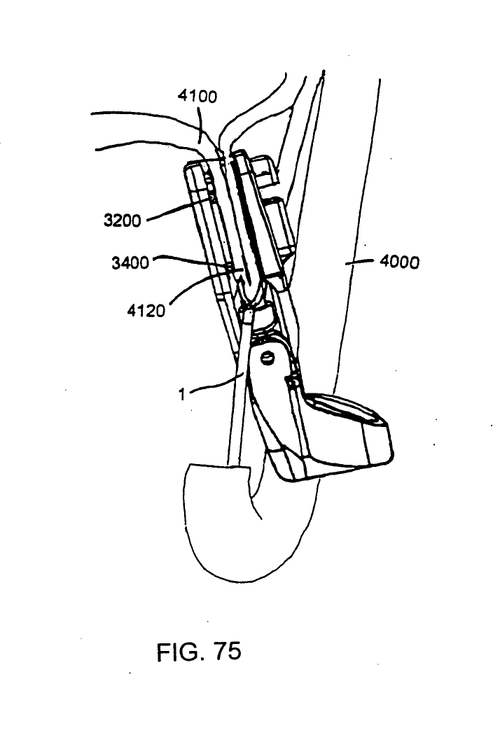

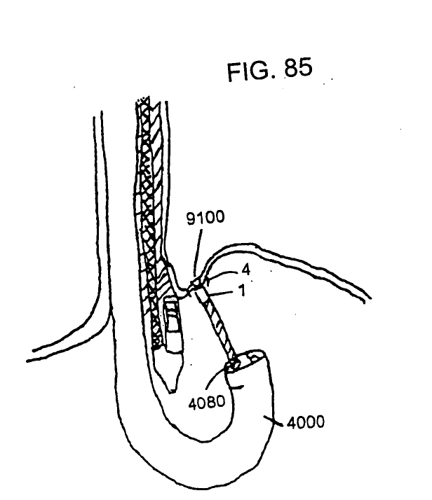

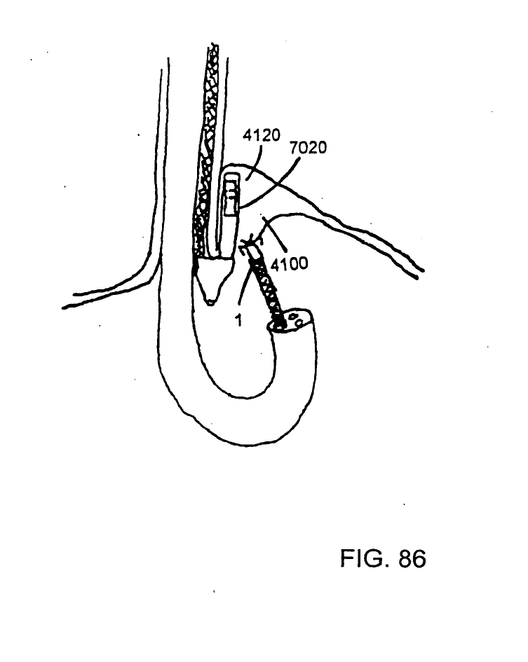

In a preferred embodiment, the flexible tissue retractor 1 is an endoscopic device that is passed through the working channel of a flexible endoscope 4000. Use of such an endoscopic retractor 1 is explained in further detail below, in particular, with regard to FIGS. 72 to 88. In such a procedure, the retractor 1 is used to hold esophageal or any other gastrointestinal tissue 4100, 9100 so that it can be moved or manipulated in some way. As the retractor 1 is passed through one of the working channels 4080 of an endoscope 4000 (see, i.e., FIG. 74 or 75), the needles 4 are in a fully retracted position in the tip 5. Once the tip 5 is set into place, it is pushed against the tissue 4100, 9100. Preferably, the tip 5 has the anchoring spike 62 to help locate a desired retraction location on the tissue and place the tip 5 at the desired location. Then, the needles 4 are actuated to extend out of the tip 5 and pierce the tissue 4100, 9100. As the needles 4 extend into and curl around the tissue 4100, 9100, it is retained securely. See FIGS. 85 and 86. Now, the tissue 4100, 9100 can be manipulated as required. To release the tissue 4100, 9100, the needles 4 need merely be retracted back into the tip 5. Because the needles 4 are pre-formed into a substantially rigid arcuate shape, they retain this shape through repeated retractions/deployments.

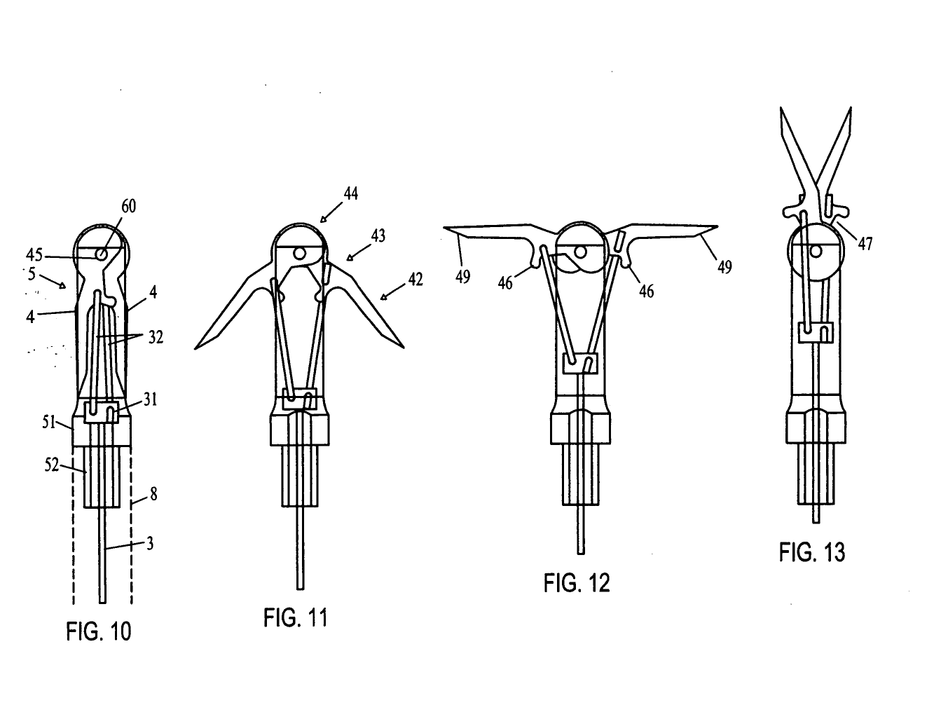

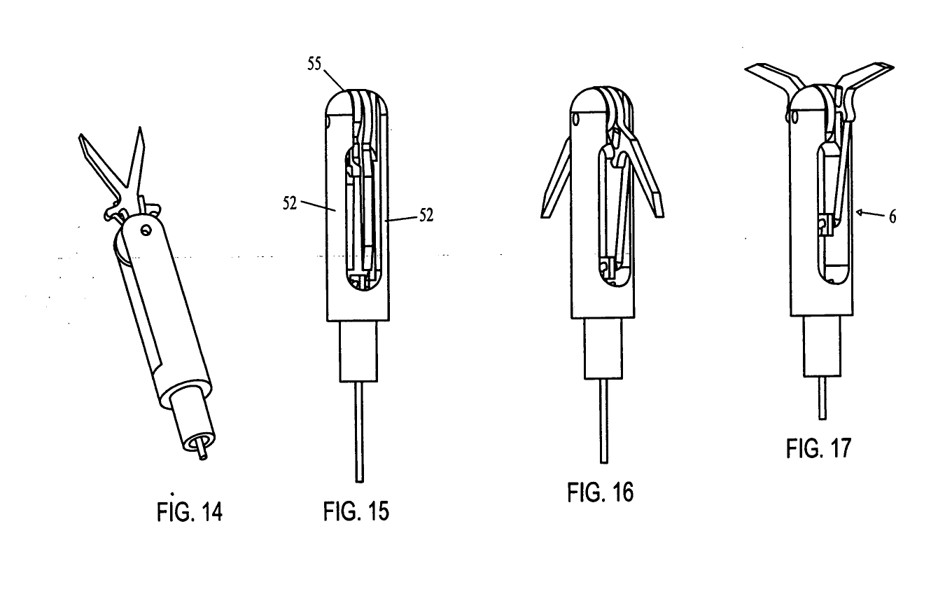

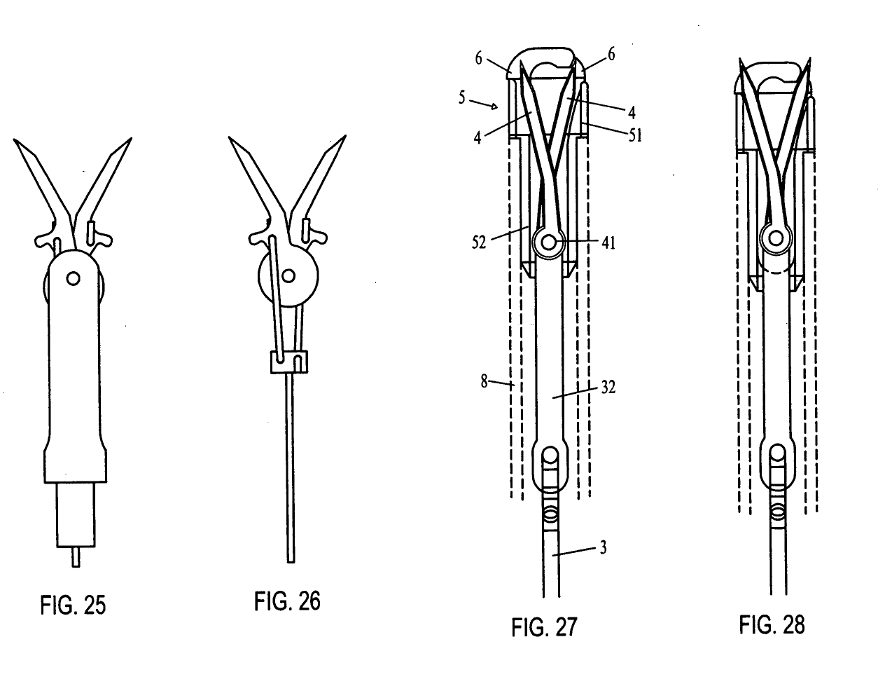

FIGS. 10 through 25 illustrate a second embodiment of a flexible tissue retractor according to the invention.

A distal tip 5 is assembled at a distal end of an outer jacket 8 (shown only diagrammatically with dashed lines). Preferably, the distal tip 5 is fastened to the distal end of the outer jacket 8. For example, the distal tip 5 can have a mushroom-shape with a head 51 and a cylindrical body 52 radially smaller than the head 51. The body 52 can be slidably inserted into the hollow distal end of the outer jacket 8 and fastened thereto using any fastening measure. For example, the body 52 can be welded, heat-shrunk, melted, or glued into the outer jacket 8 or the body can have a male thread that is screwed into a female thread disposed on the interior surface of distal end of the outer jacket 8. The connection may be reversed as well.

The distal tip 5 may be formed, for example, as a single piece or from two half-pieces secured together. If the distal tip 5 is constructed from two halves, then they can self-lock. Accordingly, the tip halves can each be formed with two holes and two pins, for example, with the holes and pins each respectively fitting into corresponding pins and holes formed in the other tip half to fix the two tip halves together. The tip halves need not be mirror-opposite. They can be identical with respect to the shape of the holes and pins. Of course, any similar fastening device, or combinations thereof, can be used to lock the tip halves to one another, i.e., screws, rivets, catch tabs and slots, and/or catch cylinders and holes.

The distal tip 5 defines a window 6 at each side thereof for receiving a respective one of the needles 4. The needles 4 are, preferably, made of steel, and, therefore, are substantially rigid.

The needles 4 can also be made of ceramic, such as alumina or zirconia, or of a polymer, such as ULTEM® or a liquid crystal polymer.

FIGS. 11 to 25 are views with various cut-away portions showing the components in the interior of the distal tip 5.

The hidden view of FIG. 10 illustrates the preferred connection of the needles 4 to a columnar actuation rod 3. This connection includes a linkage 31 connected to a distal end of the actuation rod 3 and to the proximal end of two tie rods 32, each of which is respectively connected to one of the two needles 4. The connection is best seen in FIGS. 11 to 13, 17, 18, and 24. In particular, FIG. 17 shows a preferred embodiment for individually connecting the tie rods 32 to the needles 4 and to the linkage 31.

The linkage 31 is fixedly connected to the distal end of the actuation rod 3. It can be integral with the rod 3 or it can be fastened in any manner to the distal end of the rod 3. For example, the distal end of the rod 3 can have non-illustrated male threads of a screw and the linkage can have a non-illustrated bore at the proximal side thereof with interior female threads corresponding to the male threads of the rod 3. Thus, to attach the two components together, all one must do is screw the linkage 31 onto the rod 3.

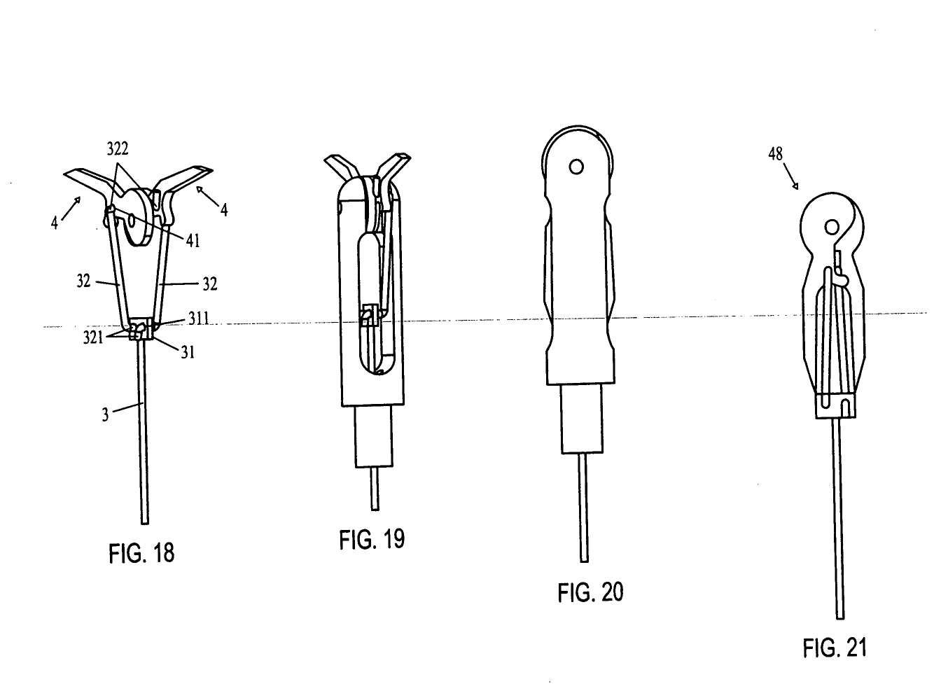

While the tie rods 32 can be fixedly connected to the linkage 31 and to the needles 4, it is preferred to have each end of the tie rods 32 be movably connected thereto. In particular, if the linkage 31 is formed with two bores 311 (see FIG. 18), then the proximal end of the tie rods 32 can be inserted, respectively, in each of the bores 311. Likewise, if each of needles 4 is formed with a bore 41, then the distal end of each tie rod 32 can be inserted therein.

The needles 4 are connected movably to the actuation rod 3, preferably, pivotally with respect to the rod 3. The tie rods 32 can be crimped, bent, or otherwise fastened to the actuation rod 3 so long as the needles 4 can move relative to the rod 3. The preferred embodiment of the retractor 1 has two needles 4. However, the number of needles can be reduced or expanded to suit particular needs.

While various connection measures can be taken to allow movement of the tie rods 32 with respect to the linkage 31, one preferred embodiment provides the tie rods 32 with an S-shaped or a Z-shaped proximal bend 321. A proximal portion of the bend 321 runs relatively parallel to the longitudinal extent of the distal tip 5, a middle portion of the bend 321 runs relatively perpendicular to the longitudinal extent of the distal tip 5, and a distal portion of the bend 321 runs relatively parallel to the longitudinal extent of the distal tip 5. Similarly, the distal end of each tie rod 32 has an S-shaped or Z-shaped distal bend 322. A proximal portion of the bend 322 runs relatively parallel to the longitudinal extent of the distal tip 5, a middle portion of the bend 322 runs relatively perpendicular to the longitudinal extent of the distal tip 5, and a distal portion of the bend 322 runs relatively parallel to the longitudinal extent of the distal tip 5. Another exemplary embodiment of the bend 321, 322 can be U-shaped.

As set forth herein, the phrase “relatively parallel” means that the longitudinal extent of the feature is more aligned with a longitudinal extent of an indicated feature than perpendicular to that indicated feature and the phrase “relatively perpendicular” means that the longitudinal extent of the feature is more perpendicular to a longitudinal extent of an indicated feature than parallel to that indicated feature.

In FIGS. 10, 15, 20, and 21, the needles 4 are in a stowed position. As can be seen best in FIG. 10, when the needles 4 are in the stowed positions, the tie rods 32 are relatively parallel to the longitudinal axis of the distal tip 5 or are slightly inclined inward with respect to the longitudinal axis of the distal tip 5—the proximal end of the tie rods 32 being further from the longitudinal axis of the distal tip 5 than the distal end of the tie rods 32.

In FIGS. 11, 12, 16 to 19, and 22 to 24, the needles 4 are partially deployed or extended in various different orientations of extension. As can be seen in the progression of FIGS. 11, 12, and 13, and of FIGS. 15, 16, and 17, and of FIGS. 20, 22, 23, and 25, as the rod 3 moves distally, the needles 4 progressively deploy towards their fully extended position, shown in FIGS. 13, 14, 25, and 26. As the deployment movement occurs, the distal ends of the tie rods 32 move away from the longitudinal axis of the distal tip 5 and, then, back towards this axis. Such out-and-in movement is due to the rotating connection of the needles 4 with respect to the distal tip 5. The entirety of the needle movement is referred to as selective movement because actuation of the needles 4 is selected by a user anywhere between the fully retracted and fully extended positions.

As best seen in the progression of FIGS. 10 through 13, the needles 4 each rotate about a similar rotation axis. The needles 4 each have a piercing portion 42, a control portion 43, and a fastening portion 44. See FIG. 11.

The piercing portion 42 is configured so that, when fully deployed (see FIG. 13), it pierces the tissue to be retracted without slicing or cutting the tissue. The piercing portion 42 has a needle end 49.

The control portion 43 is secured to a respective distal end of the tie rod 32 so that, when the tie rod 32 moves relatively parallel to the longitudinal axis of the distal tip 5, the needle 4 moves between the stowed position (FIG. 10) and the deployed position (FIG. 13). To cause this relative movement, the distal tip 5 is provided with a cylindrical axle 60 and the fastening portion 44 is formed with a bore 45 having a shape substantially corresponding to the outer shape of the axle 60. Thus, when the needles 4 are assembled next to one another between the prongs 52 of the tip 5 (see FIG. 15), and the axle 60 is inserted through the bores in each of the prongs 52 and through the bores 45 of each of the fastening portions 44 as well, the needles 4 are securely attached to the tip 5. Nonetheless, because the bores 45 are slightly larger than the axle 60, the needles 4 are able to rotate about the axle 60. The tie rods 32 are provided to limit and control such rotation about the axle 60. As used herein, the phrase “slightly larger” means that the bores 45 are large enough to not rub against the exterior surface of the axle 60 with sufficient force to, thereby, prevent movement of the needles 4. The control portion 43 has a blocking finger 46.

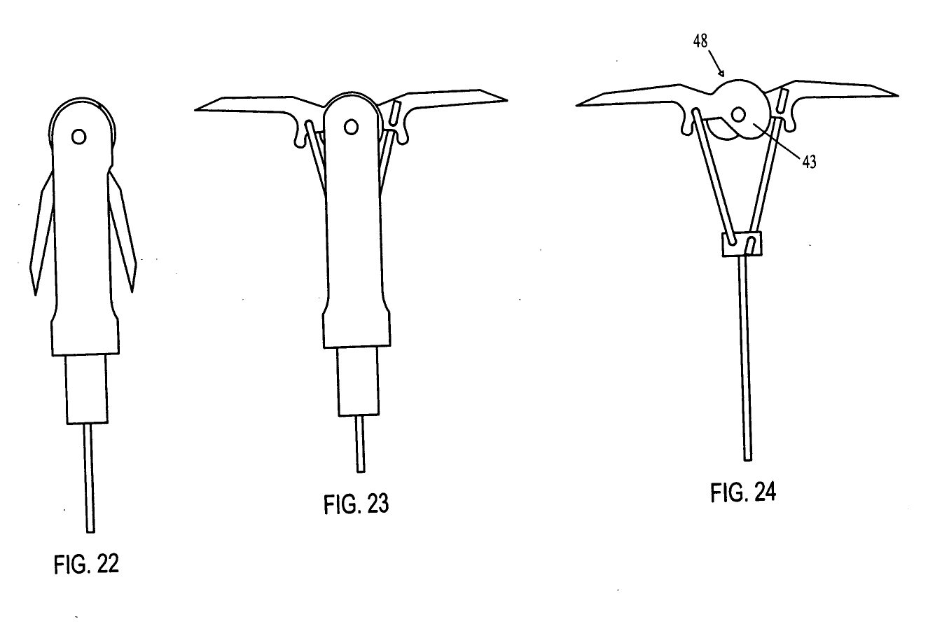

The needles 4 should only move along a single arc segment as shown in FIGS. 10 to 13, for example. To limit rotation of the needles within the arc segment, the proximal side of the blocking finger 46 is formed with a cutout 47, shown particularly well in FIG. 13. This cutout 47 acts as a stop for preventing movement in one rotation direction about the axle 60. More specifically, the needle 4 on the left of FIG. 10 is positioned in front of the needle 4 on the right of FIG. 10. Only clockwise rotation of the left needle 4 is desired and only counter-clockwise rotation of the right needle is desired. To prevent opposite rotation, the S- or Z-shape of the distal end of each tie rod 32 is formed with a middle portion sufficiently long enough to contact the proximal side of a respective blocking finger 46 when the needles 4 are approximately in the stowed position, for example, at least when the needles 4 are in the position shown in FIG. 10. To prevent this stop (formed by the middle and distal portions of the S-shape) from hitting the control portion 43 and, thereby, hindering rotation of the needle 4 about the axle 60, the distal half 48 of the control portion 43 is curved. (The curve of the distal half 48 is best shown in FIGS. 21, 24, and 26.) Thus, as the actuation rod 3 is moved, for example, from the position shown in FIG. 21 to the position shown in FIG. 24 and, then, to the position shown in FIG. 26, the stop traverses around the distal half 48 of the needle 4 but does not contact the needle 4.

The limit of needle motion (i.e., the fully deployed position) is defined by the presence of a proximal limit device (for example, in the handle shown in FIGS. 57 to 62), which prevents movement of the rod 3 past a given point. Thus, if the rod 3 can only move a given distance in a distal direction, the tie rods 32 will, likewise, only move the given distance in that direction and, therefore, only rotate the needles 4 to the position shown in FIGS. 13, 14, 25, and 26, for example. However, if movement of the rod 3 is unhindered, the length of the tie rods 32 defines the ultimate upper limit of needle motion because, in such a case where the needles are longitudinally fixed by the axle 60, the distal ends of the tie rods 32 can only move to a position directly opposite the actuation rod 3. As will be indicated below, this is not a preferred position because the needles would almost form a single needle structure directly opposite the rod 3 and, therefore, create only one piercing hole in the tissue. In such a case, as the needles 4 were rotated back towards the stowed position from the fully deployed position, the single hole would tear apart—a condition that is not desired.

Providing the blocking finger 46 at each control portion 43 also gives the configuration a second advantage—it prevents piercing of the tissue by the needles past a desired depth in the tissue that is to be retracted. Specifically, the blocking finger 46 acts as a stop preventing the piercing portion 42 from entering the tissue further than the distal side of the blocking finger 46. See FIGS. 12 and 13. If the piercing portion 42 is formed to have a short enough longitudinal extent that full penetration of the piercing portion 42 will not cause undesirable injury, then the blocking finger 46 can be removed and, instead, the distal-most surface 55 of the tip 5 would act as the stop limiting penetration of the needle 4.

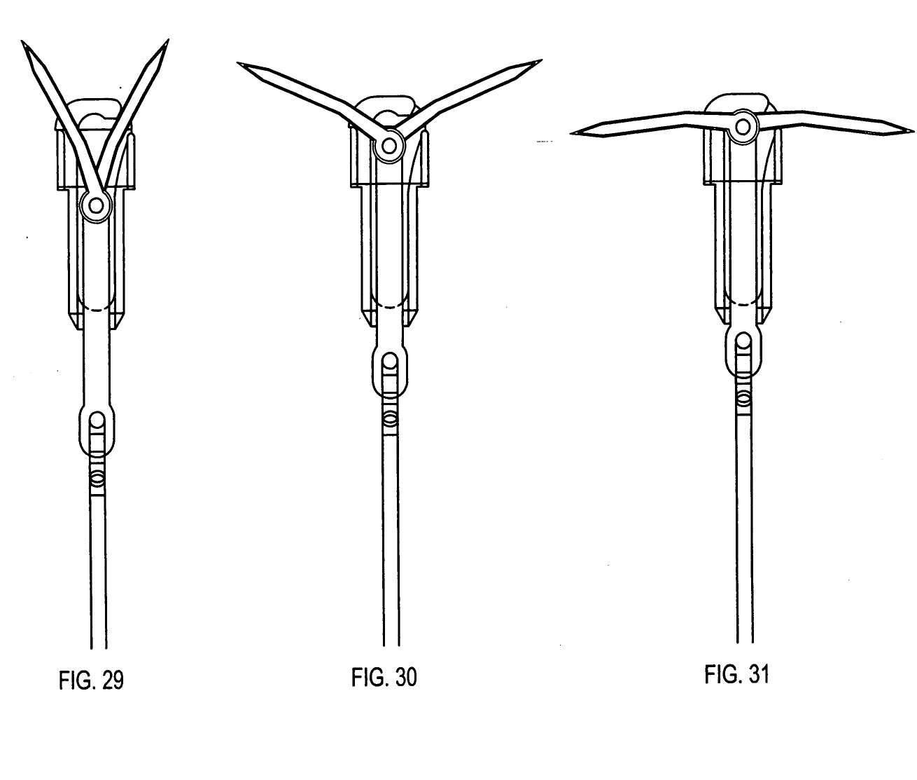

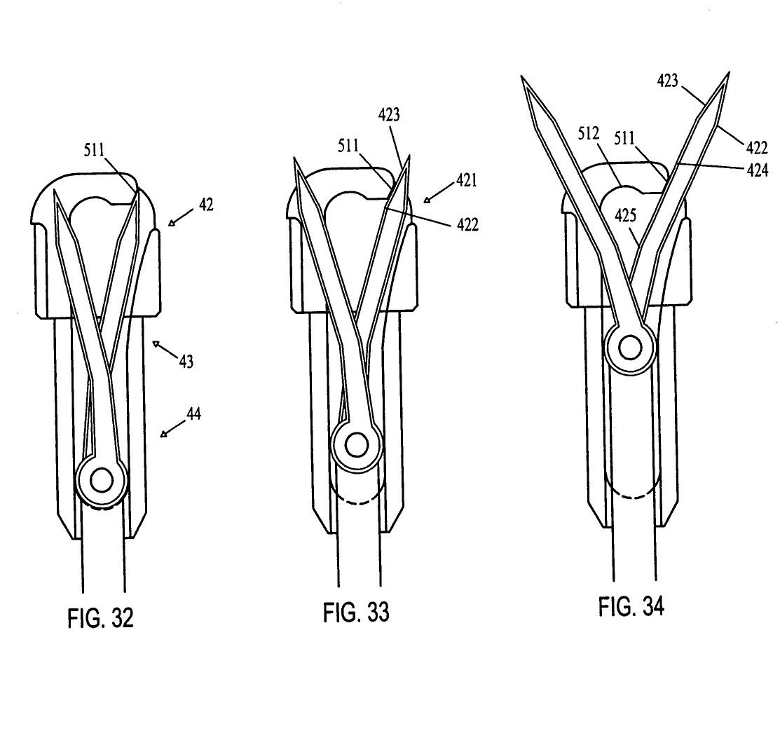

FIGS. 27 through 46 illustrate a third embodiment of a flexible tissue retractor according to the invention.

A distal tip 5 is assembled at a distal end of an outer jacket 8 (shown only diagrammatically with dashed lines). Preferably, the distal tip 5 is fastened to the distal end of the outer jacket 8. For example, the distal tip 5 can have a mushroom-shape with a head 51 and a cylindrical body 52 radially smaller than the head 51. The body 52 can be slidably inserted into the hollow distal end of the outer jacket 8 and fastened thereto using any fastening measure. For example, the body 52 can be welded, heat-shrunk, melted, or glued into the outer jacket 8 or the body can have a male thread that is screwed into a female thread disposed on the interior surface of distal end of the outer jacket 8. The connection may be reversed as well.

The distal tip 5 may be formed, for example, as a single piece or from two half-pieces secured together. If the distal tip 5 is constructed from two halves, then they can self-lock. Accordingly, the tip halves can each be formed with two holes and two pins, for example, with the holes and pins each respectively fitting into corresponding pins and holes formed in the other tip half to fix the two tip halves together. The tip halves need not be mirror-opposite. They can be identical with respect to the shape of the holes and pins. Of course, any similar fastening device, or combinations thereof, can be used to lock the tip halves to one another, i.e., screws, rivets, catch tabs and slots, and/or catch cylinders and holes.

The distal tip 5 defines a window 6 at each side thereof for receiving a respective one of the needles 4. The needles 4 are, preferably, made of steel, and, therefore, are substantially rigid. The needles 4 can also be made of ceramic, such as alumina or zirconia, or of a polymer, such as ULTEM® or a liquid crystal polymer.

FIGS. 27 through 46 are views with various cut-away portions showing the components in the interior of the distal tip 5.

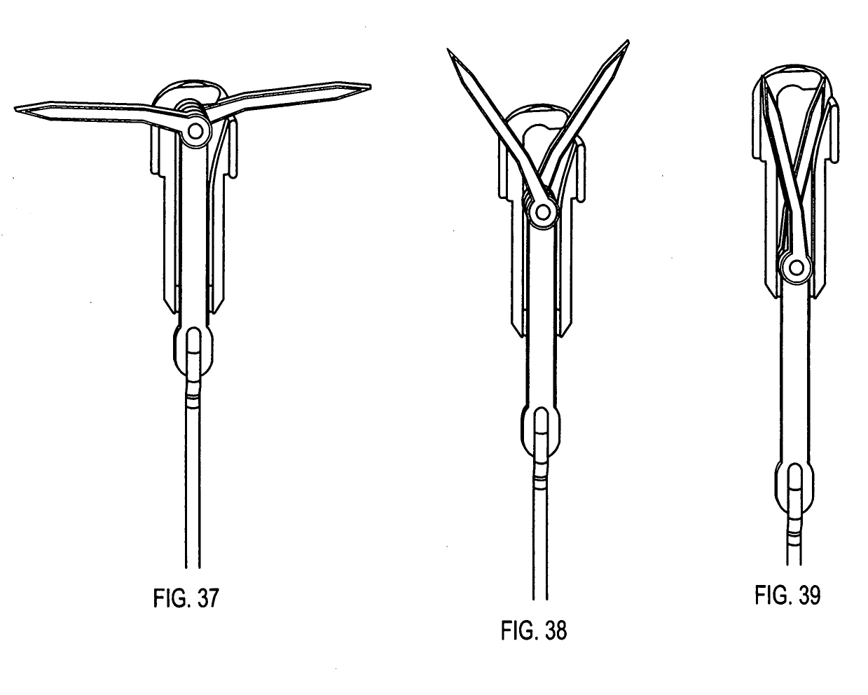

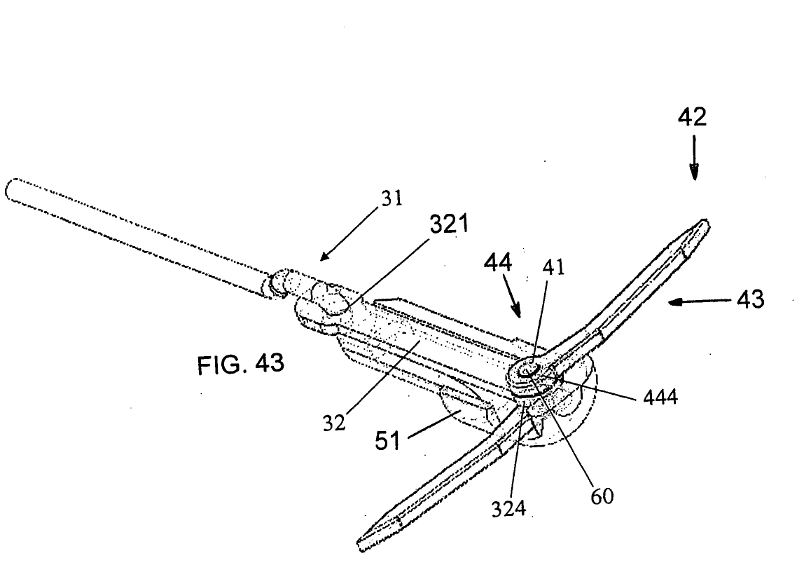

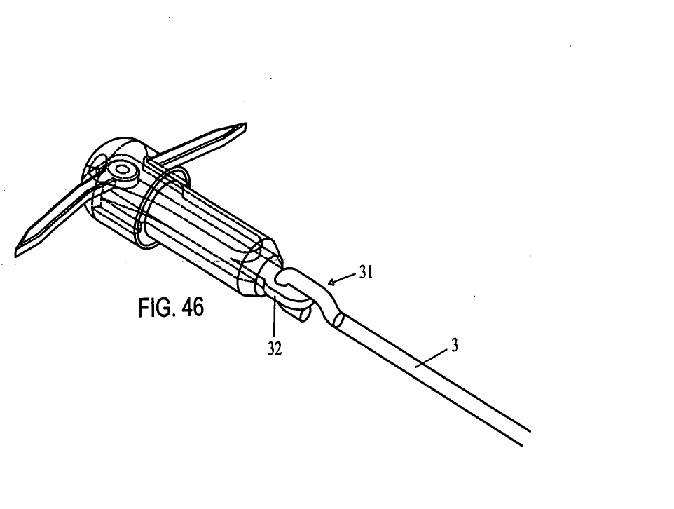

The elevational and partially cross-sectional view of FIG. 27 with half of the distal tip 5 removed illustrates the preferred connection of the needles 4 to a columnar actuation rod 3. This connection, as is best shown in the perspective view of FIG. 46 (see also FIGS. 37, 38, 39, and 43), includes a linkage 31 connected to a distal end of the actuation rod 3 and a tie rod 32. The tie rod 32 is connected to each of the two needles 4. FIG. 43 also shows the preferred embodiment for connecting the tie rod 32 to the needles 4 and to the linkage 31. The linkage 31 can be fastened in any manner to the distal end of the rod 3. The linkage 31 can be fixedly connected to or integral with the distal end of the actuation rod 3. For example, the distal end of the rod 3 can be welded to the linkage 31. It is preferable, however to connect the tie rod 32 movably to the linkage 31. In particular, if the tie rod 32 is formed with a bore 321, then the linkage 31 can be inserted movably in the bore 321. Specifically, if the linkage 31 has a C-shape as shown in FIG. 43, then the top of the “C” can be easily inserted into the bore 321 of the tie rod 32 to movably hook the rod 3 to the tie rod 32 in a manner that is not easily removable, especially when the outer jacket 8 is present.

The needles 4 can be fastened in any manner to the distal end of the tie rod 32. More specifically, each of needles 4 can be formed with a bore 41 at a proximal end thereof. Also, the distal end of the tie rod 32 can be formed with a non-illustrated bore that is axially aligned with the two bores 41 of the needles 4. Then, when the needles 4 are placed on opposing sides of the distal end of the tie rod 32 (or on the same side thereof), an axle 60 can be inserted through the three aligned bores to connect the three components together fixedly in the longitudinal direction but freely rotatable with respect to one another about the axis of the axle 60.

The axle 60 can be in one piece and integral with or attached to one half of the distal tip 5 or the axle 60 can be in two pieces and integral with or attached to each half of the distal tip 5. Alternatively, the axle 60 can be integral with the tie rod 32 and project from one or more sides thereof. It is preferable for the axle 60 to extend from one inner side of a first half of the distal tip 5 to the other inner side of a second half of the distal tip 5 so that there is no chance for the needles 4 to fall off the axle 60 at any time during use (removal of the needles 4 is envisioned only for assembly, and disassembly if desired).

Based upon the above connection, the needles 4 can move with respect to the actuation rod 3. The preferred embodiment of the retractor 1 has two needles 4. However, the number of needles can be reduced or expanded to suit particular needs. For example, if the tie rod 32 has a triangular cross-section, three needles 4 can be used, and if the tie rod 32 has a square cross-section, four needles 4 can be used.

In FIGS. 27, 32, 39, 44, 45 the needles 4 are in a stowed position. As can be seen best in FIG. 39, when the needles 4 are in the stowed position, the tie rod 32 and the proximal end of the needles 4 are relatively parallel to the longitudinal axis of the distal tip 5.

In FIGS. 28 to 30, 33 to 35, and 38 the needles 4 are partially deployed or extended in various different orientations of extension. As can be seen in the progression of FIGS. 32 to 36 and of FIGS. 39 to 37 (reverse order), as the rod 3 moves distally, the needles 4 selectively and progressively deploy from their stowed position towards their fully extended position (see FIGS. 31, 36, 37, 40 to 43, and 46). The entirety of the needle movement is referred to as selective movement because actuation of the needles 4 is selected by a user anywhere between the fully retracted and fully extended positions.

As shown in FIG. 32, the needles 4 each have a piercing portion 42, a control portion 43, and a fastening portion 44. While various connection measures can be taken to allow movement of the needles 4 with respect to the tie rod 32 and/or to the linkage 31, one preferred embodiment provides a fastening portion 324 of the tie rod 32 with a shape corresponding to the proximal end of the needles 4. In particular, as shown in FIG. 43, the tie rod 32 has a distal circular flange 324 that is sandwiched between corresponding circular flanges 444 of the fastening portions 44 of the needles 4. If the fastening portions 44 each have these corresponding circular flanges 444, then the needles 4 are tightly held between the interior of the head 51 of the distal tip 5 and the tie rod 32 to counteract lateral forces (parallel to the axis of the axle 60) and to be supported in the distal tip 5 in a very stable manner.

The movement of the needles 4 out of the distal tip 5 can be best seen in the progression of FIGS. 32 through 36.

The piercing portion 42 and the control portion 43 are configured so that, when initially deployed (see FIGS. 33 and 34), the piercing portion 42 pierces the tissue to be retracted without slicing or cutting the tissue. It is noted that the piercing portion 42 and the control portion 43 are not entirely independent from one another because a part of the piercing portion 42 controls movement of the needle 4 and a part of the control portion 43 pierces. Specifically, the distal end 421 of the piercing portion 42 is configured with a first control bend 422 and a first sloping surface 423. This bend 422 and surface 423 act as a cam follower to the first cam surface 511 that is provided at the distal end in the interior of the distal tip 5. Thus, as the distal end 421 begins to emerge from the interior of the distal tip 5, the first sloping surface 423 of the piercing portion 42 guides the tip in a relatively longitudinal direction directed distally with respect to the actuation rod 3 because the slope of this surface 423 is relatively parallel to the first cam surface 511. See FIG. 33.

As the needles 4 progress further distally, the first control bend 422 passes the first cam surface 511 and the second sloping surface 424 contacts the first cam surface 511. Because the second sloping surface 424 is relatively less parallel to the first cam surface 511 than the first sloping surface 423, the distal end 421 of the needle 4 begins to move in a relatively more perpendicular direction to the longitudinal axis of the distal tip 5. See FIG. 34.

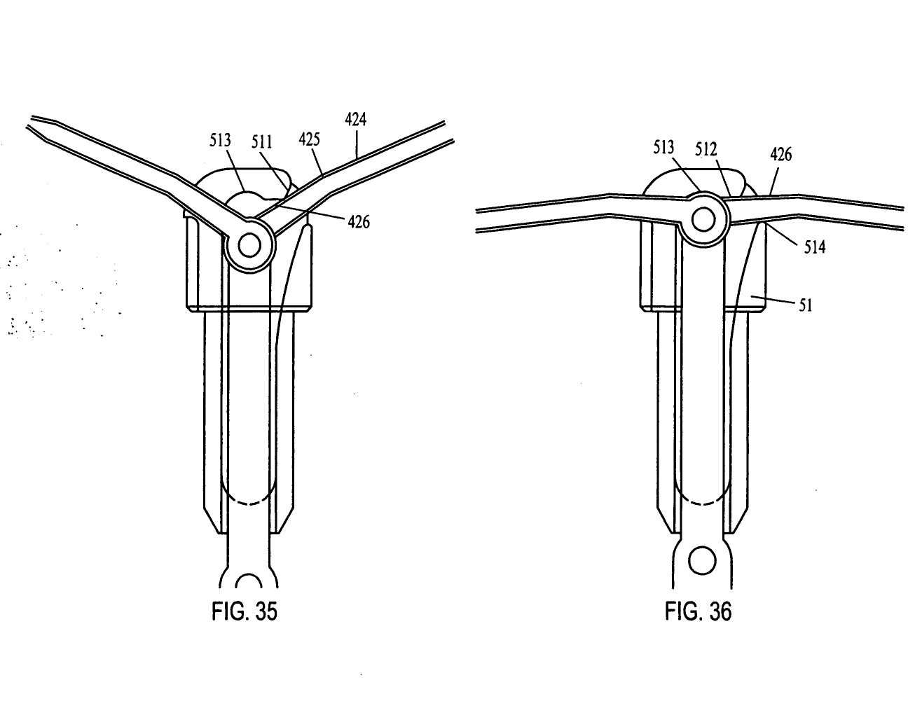

As the needles 4 progress further distally, the second control bend 425 passes the first cam surface 511 and the third sloping surface 426 (see FIG. 35) contacts the first cam surface 511. Because the third sloping surface 426 is relatively less parallel to the first cam surface 511 than the second sloping surface 424, the distal end 421 of the needle 4 begins to move in an even more perpendicular direction with respect to the longitudinal axis of the distal tip 5. See FIG. 35. Also, as the slope becomes more perpendicular, the third sloping surface 426 moves in alignment away from the first cam surface 511 and towards a second cam surface 512 (see FIG. 36), the cam surfaces 511, 512, together, forming an obtuse angle.

The limit of needle motion (i.e., the fully deployed position) is defined by the presence of a limit device, which prevents movement of the tie rod 32 past a given point. Such a limit device can be present in the handle shown in FIGS. 57 to 62. Thus, if the tie rod 32 can only move a given distance in the distal direction, the actuation rod 3 will, likewise, only move the given distance in that direction. Additionally and/or alternatively, the distal-most wall 513 of the interior of the head 51 of the distal tip 5 can be positioned to block the path of the tie rod 32. Preferably, this wall 513 is shaped with a contour corresponding to the outer contour of the tie rod 32 and/or the outer contour of the fastening portion 43 of the needles 4, but a corresponding shape is not required.

Ultimately, the distal end of the tie rod 32 contacts the distal-most wall 513 as shown in FIG. 36. At that point, it is preferred to have the third sloping surface 426 rest along and relatively parallel to the second cam surface 512. In such a position, the distal ends 421 are pointing in a direction almost or slightly greater than perpendicular to the longitudinal extent of the distal tip 5. See FIGS. 31, 36, 37, and 40 to 43.

It is further preferred to provide a third cam surface 514 of the head 51 relatively opposite at least one of the first and second cam surfaces 511, 512 so that, when the needles 4 are fully deployed, one side of the needle 4 rests against the second cam surface 512 and another side of the needle 4 rests against the third cam surface 514 without the needle 4 having play therebetween. (“Play” of the needles 4 within the openings 6 can be seen in FIGS. 32 to 35, for example.) In such a non-play configuration, the two cam surfaces 512 and 514 act to stabilize the needle 4 and support the rigidity of the needle 4 when forces act upon the needle 4 in a direction relatively perpendicular to the longitudinal extent of the needle 4. Specifically, if a force acts upon the needle 4 in a downward direction with respect to FIG. 36 (proximally), then the third cam surface 514 acts as a fulcrum and the second cam surface 512 acts as a stop counteracting such force. In particular, when the distal tip 5 is pushed in a distal direction while the needles 4 are fully deployed, the two cam surfaces 512, 514 support the needles 4 and give the needle the ability to hold more force. Likewise, if a force acts upon the needle 4 in an upward direction with respect to FIG. 36 (distally), then the second cam surface 512 (which is resting against the third sloping surface 426) acts as a stop counteracting such force. In particular, when the distal tip 5 is pulled in a proximal direction while the needles 4 are fully deployed to retract tissue, the second cam surfaces 512 support the needles 4 and gives the needle the ability to hold more force. It is noted that the widened shape of the fastening portion 44 provides additional stability and strength to the needle 4.

Movement of the needles in this third embodiment is simpler in comparison to the second embodiment.

In the third embodiment, the needles must be deployed first by moving the actuation rod 3 distally. Then, distal movement of the distal tip 5 causes the needles 4 to pierce the tissue. To effect the tissue-grasping movement—i.e., the needles 4 traversing proximally to pinch tissue between the needle 4 and the lateral sides of the distal tip 5—the actuation rod 3 must be moved proximally. Thus, the actuation rod 3 in the third embodiment must be moved, first, in a distal direction and, then, in a proximal direction.

In the third embodiment, in comparison, the needles 4 perform both of the piercing and holding movements with a single distal movement of the actuation rod 3. To allow such a two-direction movement of the needles 4, each of the needles 4 is formed with the various non-coaxial segments as set forth above.

FIGS. 47 through 56 illustrate a fourth embodiment of a flexible tissue retractor according to the invention.

A distal tip 5 is assembled at a distal end of an outer jacket 8 (shown only diagrammatically with dashed lines). Preferably, the distal tip 5 is fastened to the distal end of the outer jacket 8. For example, as shown in FIG. 54, the distal tip 5 can have a mushroom-shaped bottom 52 with a cylindrical body 51 radially smaller than the bottom 52. The bottom 52 can be slidably inserted onto the hollow distal end of the outer jacket 8 and fastened thereto using any fastening measure. For example, the bottom 52 can be welded, heat-shrunk, melted, or glued onto the outer jacket 8 or the bottom 52 can have a male thread that is screwed into a female thread disposed on the exterior surface of distal end of the outer jacket 8. The distal tip 5 may be formed, for example, as a single piece or from two half-pieces secured together. If the distal tip 5 is constructed from two halves, then they can self-lock. Accordingly, the tip halves can each be formed with two holes and two pins, for example, with the holes and pins each respectively fitting into corresponding pins and holes formed in the other tip half to fix the two tip halves together. The tip halves need not be mirror-opposite. They can be identical with respect to the shape of the holes and pins. Of course, any similar fastening device, or combinations thereof, can be used to lock the tip halves to one another, i.e., screws, rivets, catch tabs and slots, and/or catch cylinders and holes.

The distal tip 5 defines an L-shaped cutout 6 (see, in particular, FIGS. 49 and 51) at each side thereof for receiving a respective one of the needles 4. The needles 4 are, preferably, made of steel, and, therefore, are substantially rigid. The needles 4 can also be made of ceramic, such as alumina or zirconia, or of a polymer, such as ULTEM® or a liquid crystal polymer.

FIGS. 47 to 56 are views with various cut-away portions showing the components on and in the distal tip 5.

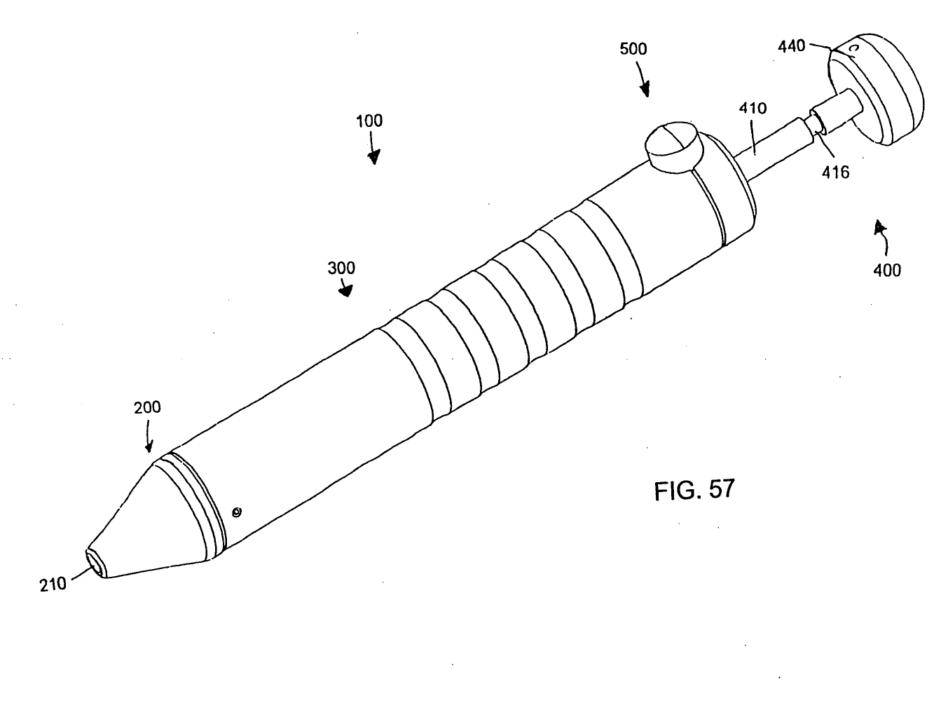

The perspective view of FIG. 56 illustrates a preferred connection of the needles 4 to a columnar actuation rod 3. This connection includes a linkage 31 (represented by a dashed line) connected to a distal end of the actuation rod 3 and to a proximal end of two tie rods 32, each of which is respectively connected to one of the two needles 4. The connection is best seen in FIG. 56 (see also FIGS. 47 to 55), which shows an embodiment for individually connecting the tie rods 32 to the needles 4 and to the linkage 31.

Specifically, the linkage 31 is fixedly connected to the distal end of the actuation rod 3. It can be integral with the rod 3 or it can be fastened in any manner to the distal end of the rod 3. For example, the distal end of the rod 3 can have non-illustrated male threads of a screw and the linkage can have a non-illustrated bore at the proximal side thereof with interior threads corresponding to the male threads of the rod 3. Thus, to attach the two components together, all one must do is screw the linkage 31 onto the rod 3, or the assembly can be reversed to have the linkage 31 screw into an internally threaded distal end of the rod 3. Alternatively, the actuation rod 3 can have a bore through which the linkage 31 is only slidably connected. In such a sliding configuration, the linkage 31 is longitudinally fixed to the rod 3 but able to rotate within the bore of the rod 3. While the tie rods 32 can be fixedly connected to the linkage 31, it is preferred to have each end of the tie rods 32 be movably connected thereto.

In particular, if the tie rods 32 are each formed with a distal bore 321, then a lateral end of the linkage 31 can be inserted, respectively, in each of the bores 321 in a slidable manner. Movement of the linkage out from the bores 321 of the tie rods 32 and actuation rod 3 can be limited by any measures, including larger ends such as rivet heads, for example.

The needles 4 are connected movably to the actuation rod 3 through the tie rods 32. The tie rods 32 can be crimped, bent, or otherwise fastened to the actuation rod 3 so long as the needles 4 can move relative to the distal tip 5. The needles 4 can be fastened in any manner to the distal end of the tie rods 32. For example, if each of needles 4 is formed with a proximal bore 41, then the distal end of each tie rod 32 can be inserted therein. While various connection measures can be taken to allow movement of the tie rods 32 with respect to the needles 4, one preferred embodiment provides the tie rods 32 with an S-shaped or Z-shaped distal bend 322. A proximal portion of the bend 322 runs relatively parallel to the longitudinal axis of the distal tip 5, a middle portion of the bend 322 runs relatively perpendicular to the longitudinal axis of the distal tip 5, and a distal portion of the bend 322 runs relatively parallel to the longitudinal axis of the distal tip 5.

In FIGS. 47 and 56, the needles 4 are in a stowed position. As can be seen best in FIG. 47, when the needles 4 are in the stowed position, the tie rods 32 are relatively parallel to the longitudinal axis of the distal tip 5 or are slightly inclined outward with respect to the longitudinal axis of the distal tip 5—the proximal end of the tie rods 32 being closer to the longitudinal axis of the distal tip 5 than the distal end of the tie rods 32.

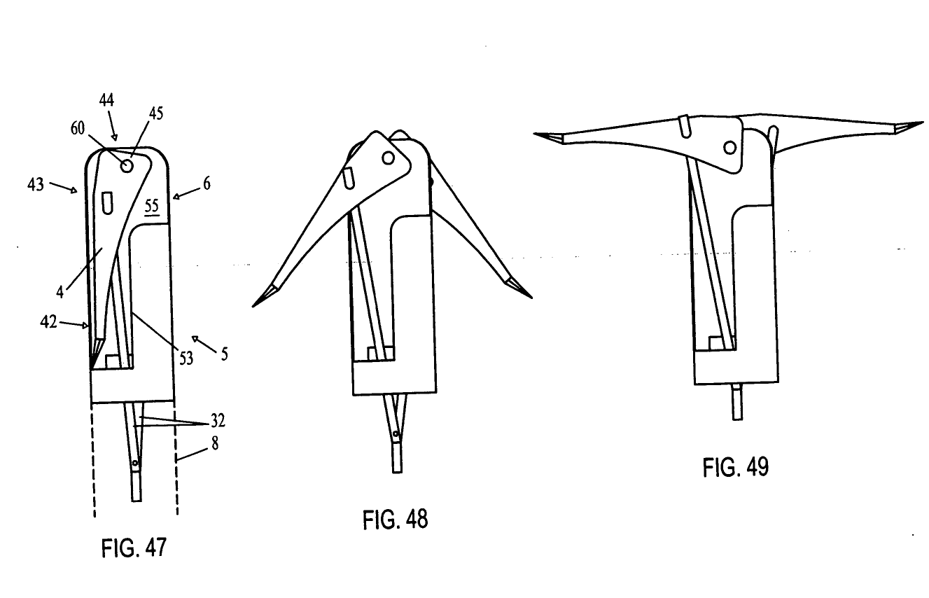

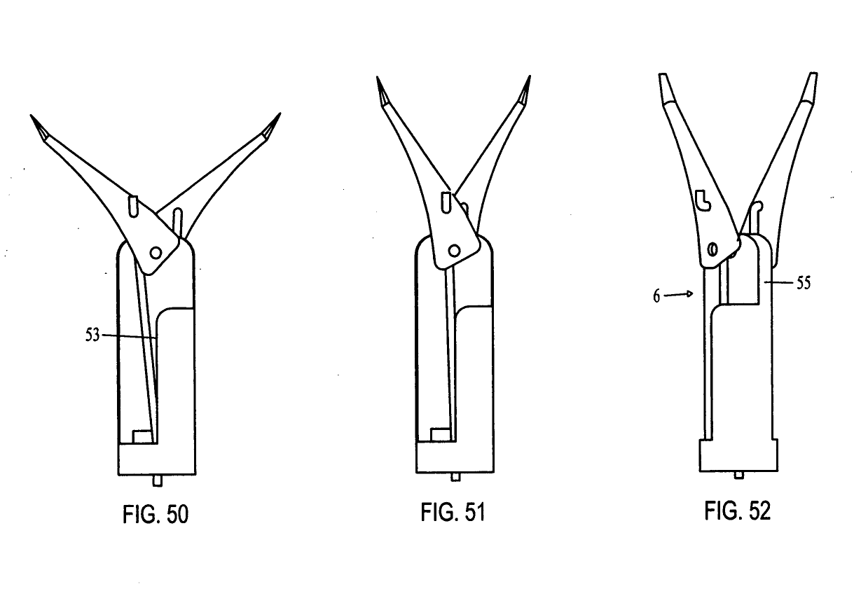

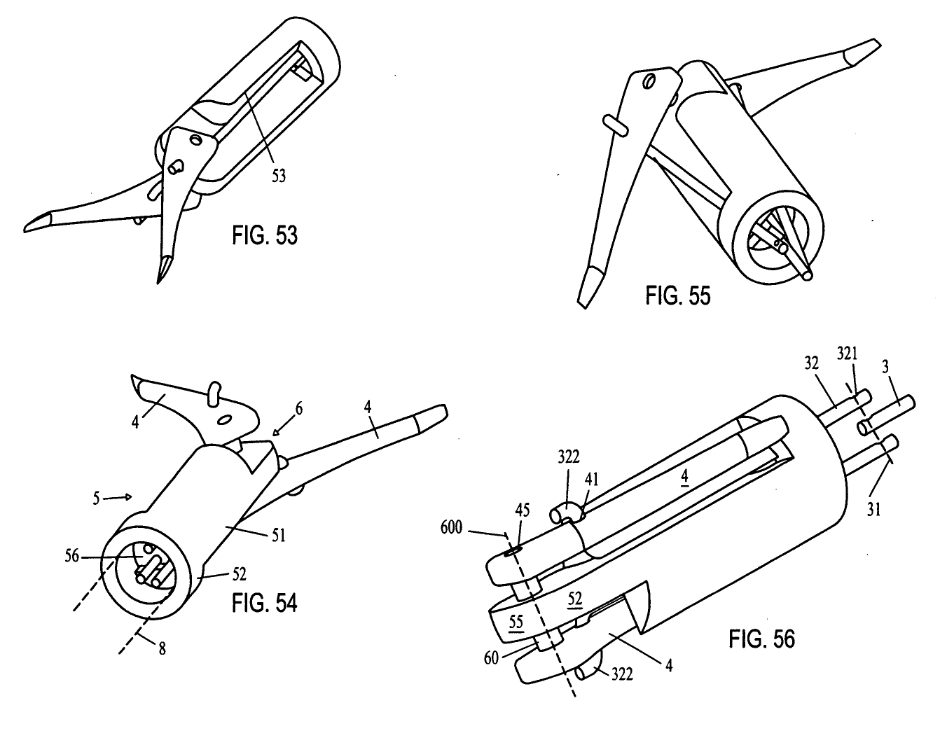

In FIGS. 48 to 50, 54 and 55, the needles 4 are partially deployed or extended in various different orientations of extension. As can be seen in the progression of FIGS. 47 to 51 and of FIGS. 56 to 53 (reverse order), as the rod 3 moves distally, the needles 4 progressively deploy towards their fully extended position, which is shown in FIGS. 51 to 53. As the selective deployment movement occurs, the distal ends of the tie rods 32 move away from the longitudinal axis of the distal tip 5 and, then, back towards this axis. Such out-and-in movement is due to the rotating connection of the needles 4 with respect to the distal tip 5. The entirety of the needle movement is referred to as selective movement because actuation of the needles 4 is selected by a user anywhere between the fully retracted and fully extended positions.

As best seen in the progression of FIGS. 47 through 51, and, in particular in FIG. 56, the needles 4 each rotate about a similar rotation axis 600. The needles 4 each have a piercing portion 42, a control portion 43, and a fastening portion 44. See FIG. 47. The piercing portion 42 is configured so that, when fully deployed (see FIG. 51), it pierces the tissue to be retracted without slicing or cutting the tissue. The control portion 43 is secured to a respective distal end of the tie rod 32 so that, when the tie rod 32 moves relatively parallel to the longitudinal axis of the distal tip 5, the needle 4 rotated between the stowed position (FIG. 47) and the fully deployed position (FIG. 51). To cause this relative selective movement, the distal tip 5 is provided with a cylindrical axle 60 and the fastening portion 44 is formed with a bore 45 having a shape substantially corresponding to the outer shape of the axle 60. Thus, when the needles 4 are assembled next to one another on opposing sides of the clevis 55 of the tip 5, and the axle 60 is inserted through the bore in the clevis 55 and through the bores 45 of each of the fastening portions 44 as well, the needles 4 are securely attached to the tip 5. Movable fastening of the axle 60 to the clevis 55 can be done by any measure that allows rotation of the needles 4 about the axle 60. Nonetheless, because the bores 45 are slightly larger than the axle 60, the needles 4 are able to rotate about the axle 60.

The tie rods 32 are provided to limit and control rotation of the needles 4 about the axle 60. The needles 4 should only move along a single arc segment as shown in FIGS. 47 to 51, for example. To limit rotation of the needles 4 within this arc segment, the cutout 6 is L-shaped with a blocking surface 53 running substantially parallel to the tie rod 32, shown particularly well in FIGS. 50 and 53. This blocking surface 53 acts as a stop for limiting a movement of each needle 4. More specifically, the needle 4 on the left of FIGS. 48 to 51 is in front of the needle 4 on the right of FIGS. 48 to 51. Only clockwise rotation of the left needle 4 is desired and only counter-clockwise rotation of the right needle is desired. To prevent opposite, undesired rotation, the blocking surface 53 is formed to prevent the distal end of the tie rod 32 from passing past the longitudinal central axis of the distal tip 5, in other words, for the left needle 4, to the right of the central axis of the distal tip 5 as viewed in FIGS. 47 to 51. As shown in FIG. 51, the tie rod 32 cannot move to the right any more than is shown therein. Accordingly, the needle 4 is limited in its movement by the arc shown in steps in FIGS. 47 to 51.

The limit of needle motion (i.e., the fully deployed position) can also be defined by the presence of a limit device that prevents movement of the actuating rod 3 past a given point. Such a limit device can be present in the handle shown in FIGS. 57 to 62. Thus, if the rod 3 can only move a given distance in a distal direction, the tie rods 32 will, likewise, only move the given distance in that direction and, therefore, only rotate the needles 4 to a final position, shown, for example, in FIGS. 51 to 53. If movement of the rod 3 is otherwise unhindered, the blocking surface 53 solely defines the ultimate upper limit of needle motion.

In this fourth embodiment, however, the distal tip 5 can have a proximal surface 56 inside the proximal end of the distal tip 5 for limiting movement of the rod 3. As shown in FIG. 54, the proximal surface 56 has holes defining ports for slidably receiving therethrough the two tie rods 32. Thus, with the proximal surface 56 present, the actuating rod 3 can only move distally until its distal end contacts the proximal surface 56. As such a point, the tie rods 32 and the needles 4 are configured to place the needle tips at the desired tissue penetrating position.

Like the second embodiment described above, providing a blocking finger on the needles 4 can prevent piercing by the needles 4 past a desired depth in the tissue that is to be retracted. Such a blocking finger would act as a stop preventing the piercing portion 42 from entering the tissue further than desired. Without such a blocking finger (as shown in FIGS. 47 to 56), the distal-most surface 55 of the distal tip 5 and/or the natural widening of the needles 4 limits penetration of the needles 4 into the tissue to be retracted.

The preferred embodiment of the retractor 1 has two needles 4. However, the number of needles can be reduced or expanded to suit particular needs.

In the above embodiments, in some instances only one side of a two-sided feature may be described or only one side may contain the reference numerals. Where the two sides are similar, identical, or symmetrical, the description can, likewise, apply to the other, non-described side and the corresponding reference numerals as well.

In all of the embodiments, the needles 4 have needle ends 49 that are, preferably, tapered.

FIG. 57 shows a handle 100 of the flexible tissue retractor 1 for controlling the extension and retraction of the needles 4 in any of the above embodiments.

The handle 100 includes a nose assembly 200, a handle assembly 300, a push-rod assembly 400, and a locking assembly 500.

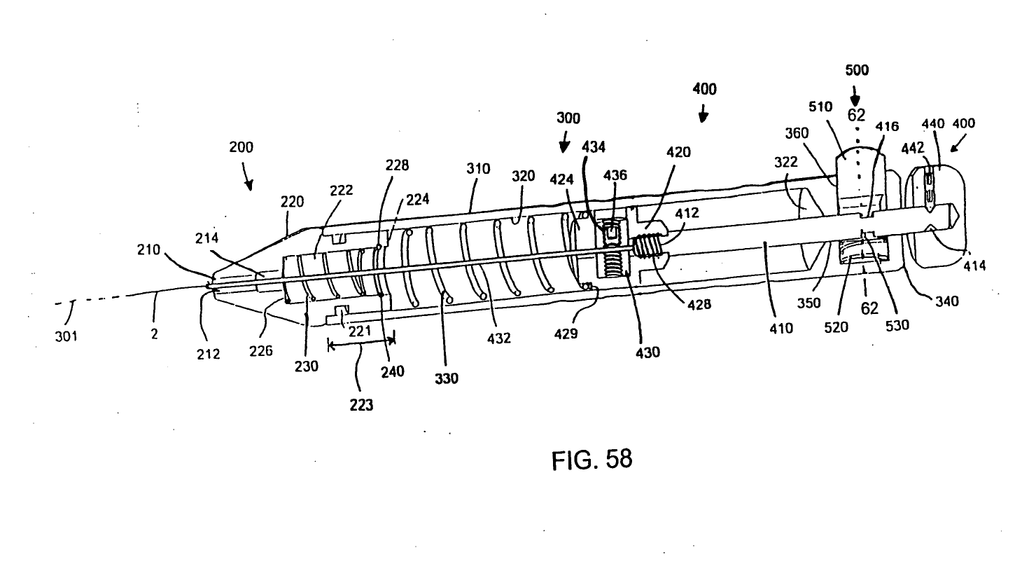

As can be seen from FIG. 58, the nose assembly 200 has a nose 220 defining a distal opening 210 and two coaxial cylindrical hollows communicating with one another including a distal hollow 212 and a proximal hollow 214. The nose 220 has a circumferential exterior setscrew groove 221. The nose 220 also defines a hollow cylindrical interior 222 (communicating with the proximal hollow 214) for receiving therein an over-stroke spring 230 (which has an exterior that is substantially cylindrical-shaped to correspond with the cylindrical shape of the interior 222 of the nose 220). The interior 222 has a distal end surface 226 and is defined at its proximal end by a proximal end surface 224 of the nose 220. Adjacent the proximal end surface 224, the interior 222 defines a groove 228, which, preferably, traverses the entire circumference of the interior 222. The groove 228 is used to seat a retaining ring 240 that holds the coil connector 10 (see FIG. 7) within the interior 222 and, thereby, retains the over-stroke spring 230 in place within the interior 222 of the nose 220.

The handle assembly 300 includes a handle body 310 defining a handle body hollow 320 extending along an axis 301 of the handle body 310. A retraction spring 330 is disposed inside the handle body hollow 320. The handle body 310 also defines, near a proximal end 340 thereof, a push-rod hollow 350 and a button hollow 360. The proximal end surface 224 of the nose 220 supports a distal end of the retraction spring 330.

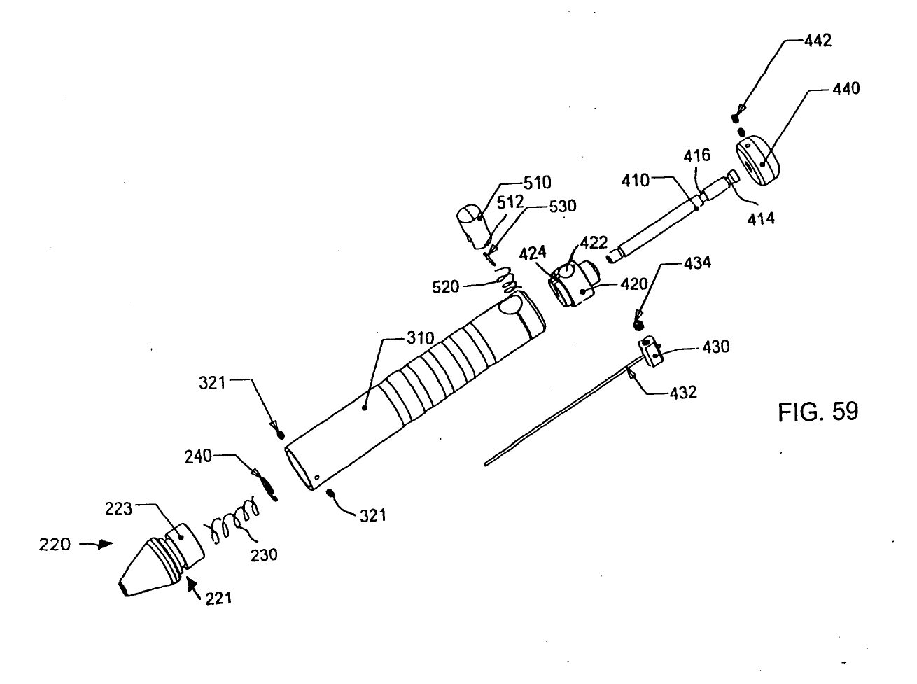

The nose 220 is connected removably to the handle body 310 by two setscrews 321 threaded into the handle body 310 and projecting through the handle body hollow 320 and into the setscrew groove 221 (see FIG. 59).

The push-rod assembly 400 is composed of a push-rod 410, a piston 420, a cross-pin 430, a cross-pin tube 432 (also referred to herein as a hypo-tube), and a knob 440. The piston 420 defines a piston hollow 422 (see FIG. 59) having a shape substantially corresponding to the exterior shape of the cross-pin 430. The piston 420 also defines a longitudinal groove or slot 424, extending from the piston hollow 422 to a distal-most end of the piston 420, the slot 424 being shaped to receive the cross-pin tube 432 therein. The cross-pin 430 has an axial bore shaped to receive therein the cross-pin tube 432. The axial bore extends along the axis 301 of the handle body 310. The cross-pin 430 also defines an interior thread 434 extending at least half way through the radial extent thereof (defined by a line orthogonal to the axis 301 when the cross-pin 430 is inserted within the piston hollow 422). Preferably, the thread 434 extends entirely therethrough so that the cross-pin 430 can be inserted into the piston hollow 422 in either orientation. A cross-pin setscrew 436 is threaded within the interior thread 432 and is tightened against the hypo-tube 432 and the actuation wire 2 (when the hypo-tube 432 with the actuation wire 2 therein are threaded into the axial bore of the cross-pin 430) to fixedly retain the two parts to the cross-pin 430.

A button 510 for locking the push rod 410 is installed in the button hollow 360, which is formed near the proximal end of the handle body 310. The button 510 defines a bore 516 having an arch shape shown in FIG. 62. The button 510 is disposed upon a button spring 520, which is also received in the button hollow 360. The button 510 has a transverse bore 512 for receiving a catch pin 530 therein. See FIG. 59. In an installed position, a contained space 514, defined by the catch pin 530 and the interior surface of the bore 516 in the button 510 enclose the push rod 410 to, thereby, retain the button 510 in the handle body 310.

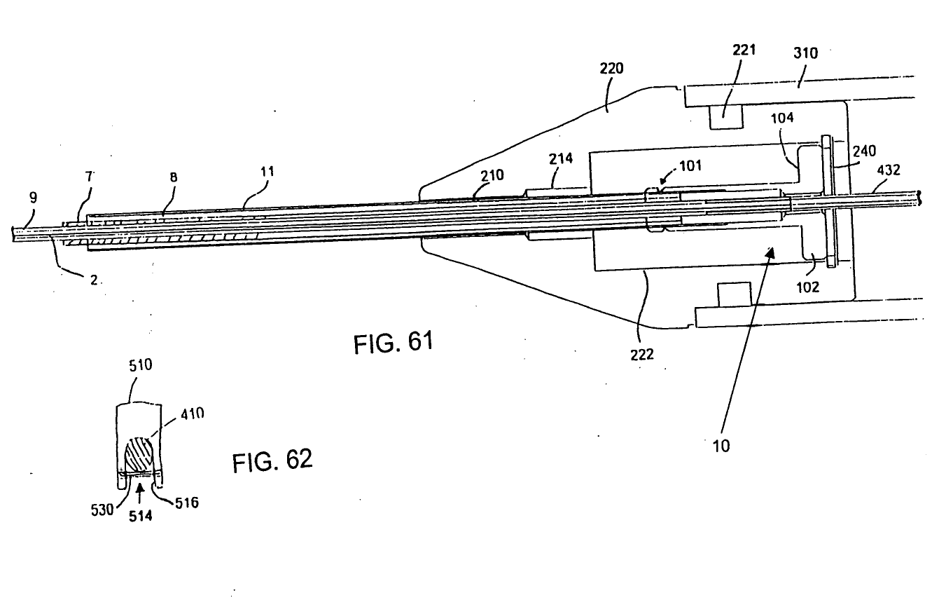

To assemble the nose assembly 200, the handle assembly 300, the push-rod assembly 400, and the locking assembly 500 reference is made to FIGS. 58 and 61, in which:
the sheath 9 is threaded over the actuation wire 2 and approximately 7 to 10 cm (3 to 4 inches) of actuation wire extends proximally from the sheath 9;
the coil winding 7 is threaded over the sheath 9;
the outer jacket 8 is threaded over the coil winding 7; and
the strain relief 11 is threaded over the outer jacket 8.

The nose 220 is threaded over the strain relief 11. The inner diameter of the distal hollow 212 is greater than the outer diameter of the strain relief 11. Therefore, there is play between the nose 220 and the strain relief 11.

Then, the over-stroke spring 230 is threaded over the strain relief 11 and is allowed to move freely into the hollow interior 222 of the nose 220. Preferably, the actuation wire 2, the coil winding 7, and the hypo-tube 432 are made of stainless steel. Thus, the sheath 9 is used to prevent the steel actuation wire 2 from rubbing against the steel coil winding 7.

Next, with regard to a cross-sectional view of an area surrounding the coil connector 10 in FIG. 61, the coil connector 10 is fixedly attached to a sub-assembly including the strain relief 11, the outer jacket 8, the coil winding 7, the sheath 9, and the actuation wire 2. Such attachment occurs, preferably, by crimping 101 the distal end of the coil connector 10. The crimping does not impart any radial force upon the actuation wire 2. Accordingly, the actuation wire 2 easily slides inside the sheath 9 and with respect to a fixed sub-assembly including the strain relief 11, the outer jacket 8, the coil winding 7, and the sheath 9.

The outer jacket 8 is, preferably, made of heat shrink tubing and is tightly shrunk on to the coil 7. Such a configuration provides a smooth outer surface for the retraction device, and also provides longitudinal stiffness to the shaft. The longitudinal stiffness is important because the needles 4 are deployed by pushing the actuation wire 2 through the shaft of the device, effectively putting the shaft in tension. If the outer jacket 8 was not shrunk tightly to the coil 7, the coil 7 could stretch and, in such a case, the device might not actuate. To that end, it is also important that the coil connector 10 be crimped to the outer jacket 8, over the coil 7, to prevent the coil 7 from stretching during actuation.

Therefore, when fully assembled, the distal hollow 212 and the proximal hollow 214 will receive therein portions of the strain relief 11, the outerjacket 8, the coil winding 7, the sheath 9, and the actuation wire 2. The strain relief 11 is also sized to extend distally out of the opening 210 approximately 10 to 15 cm (4 to 6 inches) to resist impermissible bending of the lumen 7, 8 adjacent the opening 210. In addition, the proximal hollow 214, which is wider in diameter than the distal hollow 212, is formed to receive the distal end of the coil connector 10 when the knob 440 is pressed past a position in the handle body 310 in which the needles 4 are fully extended. Such an extended position is referred to herein as “over-stroke” and will be discussed in further detail below.

To install the coil connector 10 in the nose 220, the proximal surface of the coil connector 10 is pushed to compress the over-stroke spring 230 so that the proximal surface of the coil connector 10 projects into the hollow interior 222 of the nose 220 distally past the groove 228. While holding the coil connector 10 in this position, the retaining ring 240 (which is, preferably, a C-clip with eyelets at each end of the “C” to house, for example, ends of a needle-nose-shaped pliers) is compressed and inserted into the groove 228. Because the retaining ring 240 has a radial expanse sufficient to project inward past the innermost edge of the groove 228, it acts as a proximal stop preventing the coil connector 10 from moving proximally past the retaining ring 240.

In the installed position of the coil connector 10, the actuation wire 2 projects proximally from the proximal end surface 224 of the nose 220. The hypo-tube 432 is threaded over this projecting portion until the two proximal ends thereof are aligned with one another. Then, the aligned ends are threaded into the axial bore of the cross-pin 430 at least up to the interior thread 434 and, preferably, entirely to the opposite side of the interior thread and through the other side of the axial bore. The aligned ends can project slightly out the proximal side of the axial bore because there is a distance between the proximal side of the cross-pin 430 and the distal end of the installed push-rod 410. To connect the actuation wire 2 and the hypo-tube 432 fixedly to the cross-pin 430, the cross-pin setscrew 436 is rotated inward until a sufficient force is exerted upon the hypo-tube 432 to prevent the hypo-tube 432 and the actuation wire 2 from being removed from the cross-pin 430.

The retraction spring 330 can be threaded over the actuation wire 2 before the cross-pin 430 is fastened to the hypo-tube 432 or thereafter because the internal diameter of the retraction spring 330 is similar to or greater than the diametric length of the cross-pin 430 (length of cross-pin 430 along a diameter orthogonal to the actuation wire 2 and the axis 301). The retraction spring 330 is, then, compressed sufficiently far to allow the cross-pin 430 to be inserted into the piston hollow 422, thereby, sliding the hypo-tube 432 into the longitudinal slot 424 of the piston 420. In such a position, both the over-stroke spring 230 and the retraction spring 330 are pre-loaded. The pre-compression of the retraction spring 330 is set such that the pre-compression force is great enough to always retract the needles 4 during use of the retractor device. The over-stroke spring 230 pre-compression is set such that the force required to advance the needles 4 does not exceed the pre-compression force. As such, the needles 4 will advance positively and fully before the over-stroke spring 230 begins to compress due to over stroking. In such a configuration, the retraction spring 330 is disposed between the proximal end surface 224 of the nose 220 and the distal end surface 429 of the piston 420 to bias the piston 420 in a proximal direction towards the knob 440.

The distal male threaded end 412 of the push rod 410 is screwed into female threads 428 of the proximal end of the piston 420. As such, the piston 420 form-lockingly engages the push-rod 410. A form-locking connection is one that connects two elements together due to the shape of the elements themselves, as opposed to a force-locking connection, which locks the elements together by force external to the elements. Thus, axial movement of the actuation wire 2 linearly follows axial movement of the piston 420.

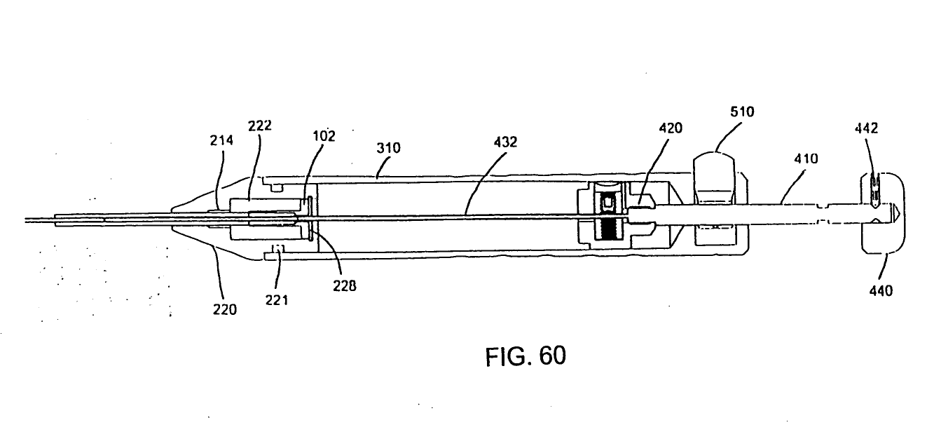

Now, the handle body 310 is distally threaded over the proximal end of the push-rod 410, over the piston 420, over the retention spring 330, and, finally, snugly over the proximal stub 223 of the nose 220 and secured thereon by at least one setscrew 321 (see FIG. 59). Before the proximal end of the push-rod 410 enters the button hollow 360, however, the button spring 520 is inserted in the button hollow 360 and the pre-assembled the button 510 (catch pin 530 inserted into the transverse bore 512 as shown in FIG. 62) is pressed against the button spring 520 to compress the spring to such an extent that the contained space 514 (between the catch pin 530 and the interior surface 516 in the button 510) is aligned approximately with the axis 301 and, therefore, with the axis of the push-rod 410. In such a position, the proximal end of the push-rod 410 will pass through the contained space 514 without substantial friction and project out of the proximal end of the handle body 310. It is noted that, to assist threading of the proximal end of the push-rod 410 into the proximal end of the handle body 310, as shown in FIGS. 58 and 60, the proximal surface 322 of the handle body hollow 320 is tapered towards the push-rod hollow 350 in the form of a funnel.

The proximal end of the push-rod 410 is formed with a circumferential set-pin groove 414 to accommodate a knob set pin 442 that fixedly connects the knob 440 to the push-rod 410. In an alternative non-illustrated embodiment, the proximal end of the push-rod 410 can be formed with a male thread that corresponds to a female thread in the knob 440 such that the knob 440 is screwed onto the push-rod 410. The push-rod 410 is also formed with a circumferential catch pin groove 416 used to capture the catch pin 530 when the push-rod 410 is pressed from a proximal position shown in FIG. 60 to a distal position shown in FIG. 58. In the proximal position, the needles 4 are retracted within the tip 5 and, in the distal position, the needles 4 are extended out of the tip 5. When the catch pin 530 is within the groove 416, the needles 4 are in the distal position and the knob 440 can only be moved slightly; such movement is permitted by the play created by the length of the groove 416 along the longitudinal extent of the push-rod 410. A user can selectively engage the button 510 to capture the push-rod 410 with the catch pin 530 or allow the push-rod 410 to move freely in the longitudinal direction by pressing the button 510 to move the catch pin 530 out of the way so that the groove 416 does not engage the catch pin 530. Accordingly, the locking function of the button 510 can be said to selectively retain the needles 4 in a given position. Of course, there can be a plurality of spaced apart grooves 416 to provide different retention positions from the position that is illustrated, for example, in FIG. 58.

It is noted that the distal end of the hypo-tube 432 rests proximal of the distal end of the coil connector 10 when the piston 420 is in a proximal-most position and rests distal to the distal opening 210 of the nose 220 when (as shown in FIG. 58) the knob 440 is pressed to a distal position such that the catch pin 530 engages the catch pin groove 416, this distal position corresponding to a position in which the needles 4 are completely extended out from the tip 5 as shown in FIG. 1, for example. The distal position shown in FIG. 58, however, is not the distal-most position of the knob 440. Such a feature is intentional because of the nature of the retractor 1 being an assembly that is intended to be inserted into a patient (in particular, through an endoscope) and, therefore, is to be curved because of the nature of the patient and the endoscope.

Geometry of the co-axially placed actuation wire 2, sheath 9, coil winding 7, and outer jacket 8 dictates that, when the outer jacket 8 and/or the coil winding 7 is bent (as it traverses through an endoscope, for example), the length of the actuation wire 2 needed to traverse the curved lumen 7, 8 increases. The actuation wire 2 is configured, therefore, to be sufficiently long such that over-stroke does not occur when the lumen 7, 8 are curved in use. However, when the coil winding 7 and the outer jacket 8 are straight, the operation of the push-rod 410 may cause over-stroke because the actuation wire 2 is longer than the lumen 7, 8 surrounding the actuation wire 2. In such a case, there arises the above-mentioned danger of needle over-extension, which could cause damage to the tissue to be retracted or to the needles 4 themselves. To compensate for such over-stroke, the nose assembly 200 is provided with the over-stroke spring 230, which is disposed in the hollow interior 222 of the nose 220. The over-stroke spring 230 is supported at its distal end by the distal end surface 226 of the hollow interior 222 and at its proximal end by the distal surface 104 of a head 102 of the coil connector 10 (see FIG. 61). Such a configuration, effectively, decouples the lumen 7, 8, 9 (and 11) from the actuation wire 2. To fully deploy the needles 4 in an over-stroke situation, the knob 440 must be pressed in more than if the lumen 7, 8 were curved. Accordingly, the over-stroke spring 230 has a k-factor tuned to allow full deployment of the needles 4 and, thereafter, to compress for further distal movement and compensate for the over-stroke. Due to this tuning of the over-stroke spring 230, the coil winding 7, through the coil connector 10, will compress against the over-stroke spring 230 and move the entire sub-assembly of the lumen 7, 8, and 9 distally and, thereby, absorb the over-stroke of the push-rod 410. Thus, the over-stroke spring 230 functions as a buffer to absorb any over-stroke of the push-rod 410 and substantially prevents any disadvantageous affects when in use. Specifically, the over-stroke spring 230 protects the needles 4 from over-extending and protects the coil winding 7 from being over extended. It is noted that, most of the time, the coil winding 7 and the outer jacket 8 will be curved during operation. Thus, the coil winding 7 and the retraction spring 330 are constructed to provide proper extension for the needles 4 in such a situation.

When pushing the knob 440, the retraction spring 330 will be compressed and the needles 4 will be extended out of the tip 5 by the actuation wire 2. After the needles 4 are extended to a certain distance, the catch-pin 530 will fall into the catch-pin groove 416 formed on the push-rod 410, thus preventing the push-rod 410 from further movement and locking the needles 4 in the deployed position (assuming that the button 510 is not being depressed). Because the needles 4 are held in the deployed position, the user is, then, free to let go of the handle without the fear of needle 4 retraction, and to use their hands for other surgical procedures until retraction of the needles 4 is desired. By pressing the button 510 down, the catch-pin 530 is forced out of the catch-pin groove 416, thereby unlocking the push-rod 410 and automatically retracting the needles 4 because the retraction spring 330 imparts a proximally directed bias to the piston 420.

By way of example only, preferred dimensions for one exemplary handle 100 are set forth in the following text. An overall longitudinal length of the handle 100 is, preferably, approximately 17 cm (6.74 inches). A preferred longitudinal length of the handle body 310 is between approximately 13 and 14 cm (5.29 inches). A preferred distance between the distal surface of the knob 440 and the proximal end surface of the handle body 310 is between approximately 2.5 and 3 cm (1.05 inches).

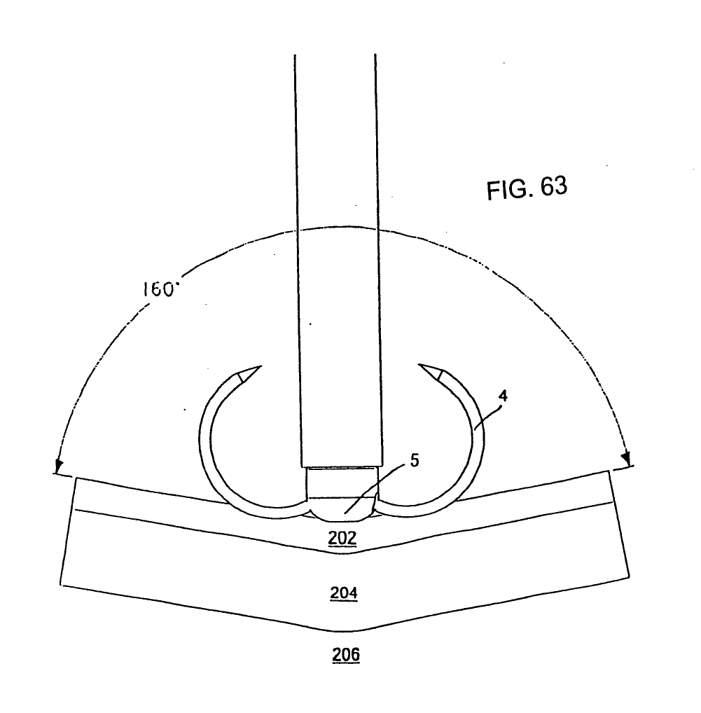

The operation of the flexible tissue retractor 1 of the invention will be described in the following text with respect to FIGS. 63 through 70.

The needles 4 are fully retracted into the tip 5 of the retractor 1 as the retractor 1 passes through the endoscope. Using the camera of the endoscope, the tip 5 is positioned at the desired location, in particular, in a selected location of the wall of the stomach. The spike 62 is used to keep the tip 5 in position once the tip 5 is advanced to contact the mucosa 202, the innermost layer of the stomach, for example.

In the treatment of Gastroesophageal Reflux Disease, it is most desirable to grasp the muscularis 204 of the stomach, an interior layer adjacent the mucosa 202. It is not desirable, for the reasons stated above, to puncture the serosa 206 of the stomach (the outermost layer). Therefore, the shaped curvature of the needles 4 is configured so that penetration will occur to a depth no greater than the muscularis 204. Such assurance is illustrated with regard to FIGS. 63 to 70.

Once in place in the stomach, the tip 5 is pushed against the mucosa 202 and the needles 4 are extended out of the tip 5 to pierce the stomach tissue with the goal of reaching the muscularis 204. The piercing depth of the needles 4 is dependent upon the degree in which the tip 5 is pressed against the mucosa.

If the tip 5 is pressed against the mucosa 202 such that the mucosa 202 presents a 160° angle to the distal face of the tip 5, as shown in FIG. 63, the needles 4 will barely penetrate the mucosa 202 or will not penetrate the mucosa 202 at all.

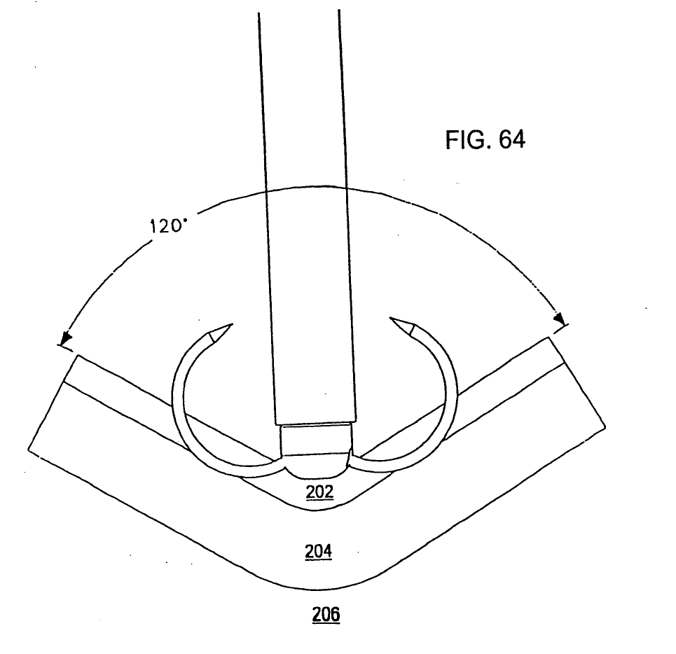

If the tip 5 is pressed against the mucosa 202 such that the mucosa 202 presents a 120° angle to the distal face of the tip 5, as shown in FIG. 64, the needles 4 will penetrate the mucosa 202 but will barely penetrate the muscularis 204 or will not penetrate the muscularis 204 at all.

If the tip 5 is pressed against the mucosa 202 such that the mucosa 202 presents a 90° angle to the distal face of the tip 5, as shown in FIG. 65, the needles 4 will penetrate the muscularis 204 sufficiently far for a proper retraction.

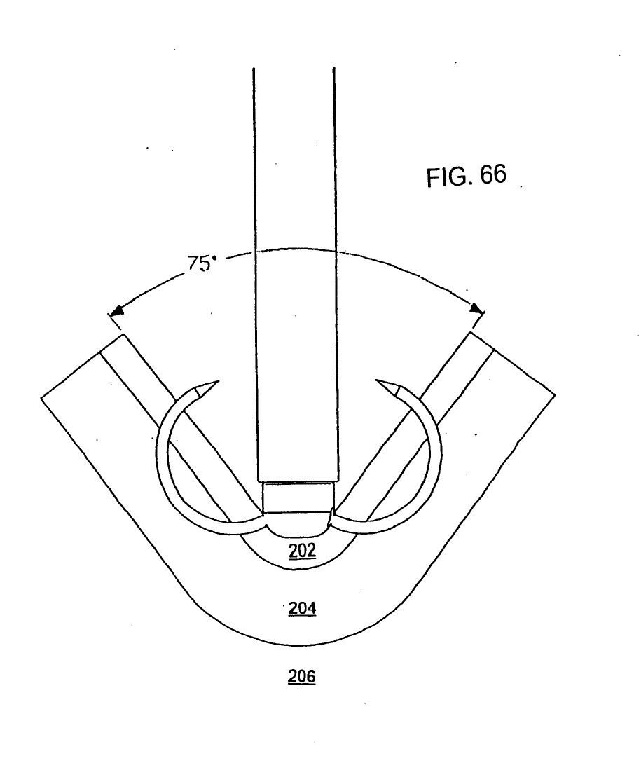

If the tip 5 is pressed against the mucosa 202 such that the mucosa 202 presents a 75° angle to the distal face of the tip 5, as shown in FIG. 66, the needles 4 will penetrate the muscularis 204 sufficiently far for a proper retraction.

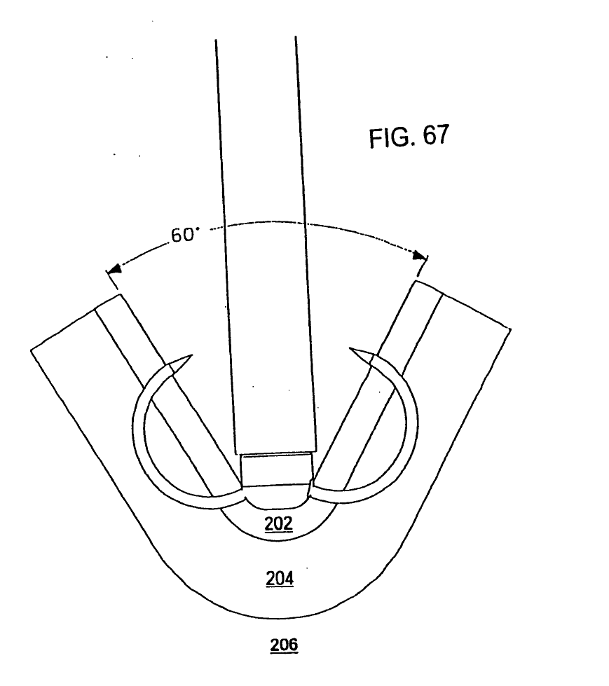

If the tip 5 is pressed against the mucosa 202 such that the mucosa 202 presents a 60° angle to the distal face of the tip 5, as shown in FIG. 67, the needles 4 will penetrate muscularis 204 sufficiently far for a proper retraction.

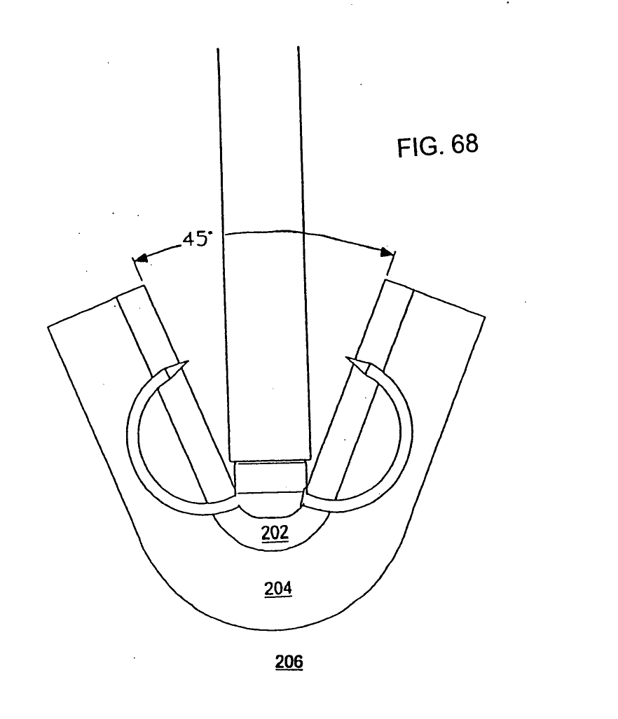

If the tip 5 is pressed against the mucosa 202 such that the mucosa 202 presents a 45° angle to the distal face of the tip 5, as shown in FIG. 68, the needles 4 will penetrate more than a majority of the muscularis 204 for a proper retraction but still far short of the serosa 206.

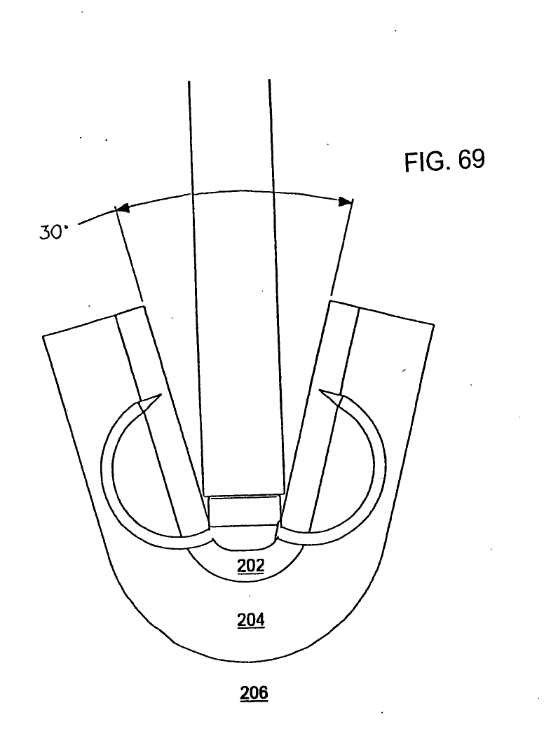

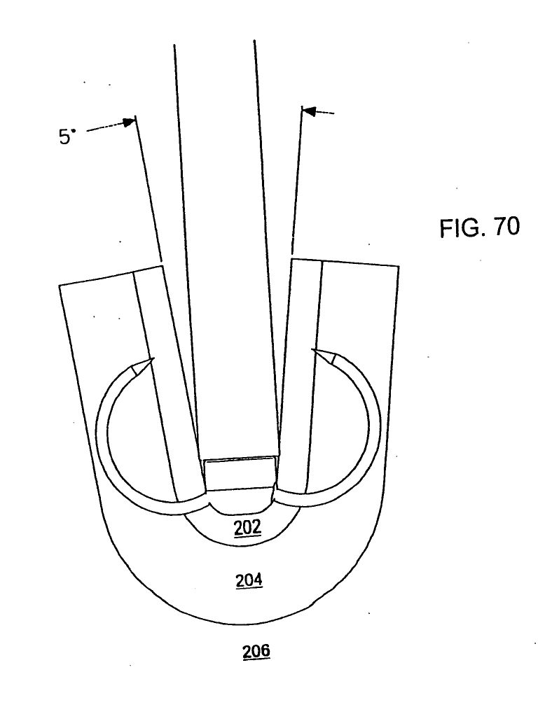

If the tip 5 is pressed against the mucosa 202 such that the mucosa 202 presents a 30° angle to the distal face of the tip 5, as shown in FIG. 69, the needles 4 will penetrate more than a majority of the muscularis 204 for a proper retraction but still not as far as the serosa 206.

If the tip 5 is pressed against the mucosa 202 such that the mucosa 202 presents a 5° angle to the distal face of the tip 5, as shown in FIG. 70, the needles 4 will penetrate more than a majority of the muscularis 204 for a proper retraction but still just before the serosa 206.

Of course, the actual degree of penetration will be dependent on the thickness of the mucosa 202 at the given retraction site and upon the respective thicknesses of the patient's stomach layers 202, 204, 206. Nonetheless, the sizing of the needles' curvature should behave as stated above for non-abnormal patients.

When retracted properly, the tissue can be manipulated or moved as required. Release of the tissue, by retracting the needles 4 back into the tip 5, occurs simply by a press of the button 510.

The needles 4 are dimensioned and shaped such that they will most likely not enter the serosa 206. As shown in FIGS. 63 to 70, no matter how hard the tip 5 is pushed against the stomach tissue, the needles 4 will stay safely within the serosa 206 and only pierce the mucosa 202 and muscularis 204—a desirable situation for treatment of GERD because the fastening clip can be implanted in a most-optimal position within the patient.

By varying the position of the needle exit locations and/or the angle of exit at the tip of the retractor 1, the needles 4 can be made to extend proximal to a plane at the tip of the retractor 1 and orthogonal to the longitudinal axis of the tip 5 or distal to the plane. Further, by varying the length and/or the radius of curvature of the needles 4, the depth of penetration in the tissue can be limited or enhanced. If the needles 4 exit proximal to the tip plane, their depth of penetration will be limited. Such an example is shown in FIG. 71. Conversely, if the needles 4 extend beyond the plane, their depth of penetration will be enhanced. If the needles 4 are longer and with a greater radius of curvature, the penetration will be enhanced, while conversely, if they are short and the radius of curvature is decreased, the penetration will be limited. By varying the combinations and dimensions of these parameters, the retractor 1 can be tailored to penetrate the tissue in different ways, allowing the grasping of specific layers of the tissue to suit the procedural requirements.

In the case of the endoscopic treatment of GERD, experience has shown that a user can tell whether or not the retraction of the wall is proper, in other words, both the mucosa 202 and muscularis 204 are retracted. The retracted stomach tissue exhibits a significantly different shape when both the mucosa 202 and muscularis 204 are retracted as compared to when only the mucosa 202 is retracted. The mucosa 202 and muscularis 204 are analogous to a bag within a bag, in that, the mucosa 202 is not truly attached to the muscularis 204 and, therefore, when only the mucosa 202 is retracted a clearly visible and sharply slanted peak is formed by the retracted mucosa 202. In contrast, when both the mucosa 202 and muscularis 204 are retracted, a clearly visible and smoothly sloped hill is formed. The visual confirmation assures that at least the muscularis 204 was grabbed and the size and shape of the needles 4 ensures a reduction of the possibility of perforating the serosa 206. The serosa 206, unlike the mucosa 202, is truly attached to the muscularis 204. It is a very thin layer and, thus, moves with the muscular layer 204.

According to a method of the present invention, the retractor 1 may be operated as follows with respect to the treatment of GERD. Turning to FIG. 72, a sleeve 3200 of a distal end effector 2020 is slidably coupled over the distal end of an endoscope 4000 and the end effector 2020 is slid proximally over the endoscope 4000. (It is noted that a sleeve 3200 is not a required feature and is merely exemplary.) The distal end of the endoscope 4000 is, then, inserted into the tracheopharangeal passage and moved through the esophagus 4140 and into the stomach 4160, with the end effector 2020 of the plication instrument mounted, approximately 20 cm back from the distal end of the endoscope 4000. The actuating handle and/or control shaft 2060 are, then, manipulated in gross to slide the distal end effector 2020 over the distal end of the inserted endoscope 4000 and into the stomach 4160, with the endoscope 4000 functioning as a guidewire for the sleeve 3200. Optionally, the endoscope 4000 may be retroflexed to look back toward to the LES 4020 of the esophagus 4140 and visualize the advancement of the end effector 2020.

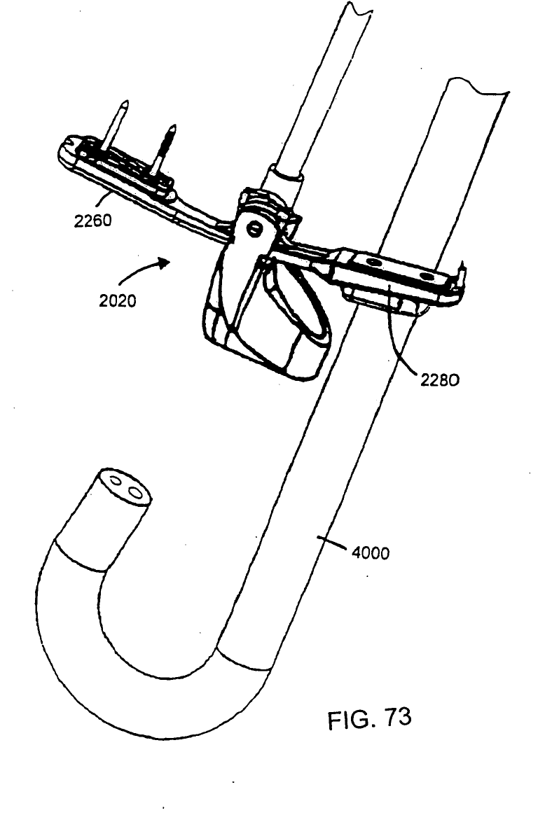

If the endoscope is retroflexed during insertion of the distal end effector 2020, the passage of the distal end effector 2020 into the stomach 4160 is performed under view of the endoscope 4000. Once the distal end effector 2020 is located in the stomach 4160, the endoscope 4000 is straightened (if it was retroflexed), and the end effector 2020 is moved distally off the endoscope 4000 such that the endoscope 4000 and clip implantation instrument are completely separated. Referring to FIG. 73, the endoscope 4000 is then, again, retroflexed and the actuating handle is operated to open the jaws 2260, 2280 of the end effector 2020.

Referring to FIG. 74, a retractor 1 according to the present invention is, then, inserted through a working channel 4080 of the endoscope 4000 and directed at target tissue 4100 one to three centimeters into the stomach 4160 adjacent the LES 4020 where the center of a plication is to be located. The retractor 1 engages the tissue 4100 and pulls the tissue 4100 back between the jaws 2260, 2280 of the end effector 2020 of the clip implantation instrument. The retractor 1 engages the deep muscle of the stomach wall, thus retracting a full thickness plication of the stomach wall between the jaws. In addition, the actuating handle and/or control shaft 2060 of the clip implantation instrument are pulled back in gross (i.e., in the direction of withdrawing the instrument) such that the jaws 2260, 2280 approach the tissue 4100 in a direction substantially parallel to the esophagus 4140. This is a highly desirable angle of approach that has been previously unattainable with endoscopic GERD treatment instruments. That is, any device that retroflexes must extend through an arc of a minimum radius. This radius is such that when retroflexed therethrough, the distal end of the device will be displaced thereby, and, thus, the end effectors will be further away from the GEJ than from a device that does not require retroflexion. It is not possible, therefore, for a retroflexed device to be both parallel to an entry path and also not displaced at least a couple of centimeters away from the entry path.

A non-illustrated proximal actuation handle is, then, operated to cause the jaws 2260, 2280 to close, as shown in FIG. 75. As a central point of the tissue 4100 is held in a fixed location between the jaws 2260, 2280 by the retractor 1 during movement of the jaws 2260, 2280, a tissue plication 4120 is formed by the jaws 2260, 2280 as the male and female parts 1200, 1400 of the fastener 1000 are brought together with the plication 4120 clamped therebetween. When the jaws 2260, 2280 are closed about the tissue plication 4120, the posts 3200, 3400 of the male part 1200 of the fastener 100, preferably, pierce the tissue 4100 through the serosal layers of the plication forming a serosa-to-serosa contact on the inside surfaces of the plication. Experimental procedures have shown that such contact results in tissue adhesion after healing such that the tissue 4100 is permanently reconfigured, i.e., even if the fastener 1000 is removed later. In this manner, a zone of reduced compliance is created about the LES 4020.

The location and size of the plication 4120 as well as the relative positions of the fastener parts 1200, 1400 are observed through the endoscope 4000. Moreover, less or more clamping pressure can be applied to the plicated tissue by control of a proximal actuation handle until full penetration by the male posts has been achieved.

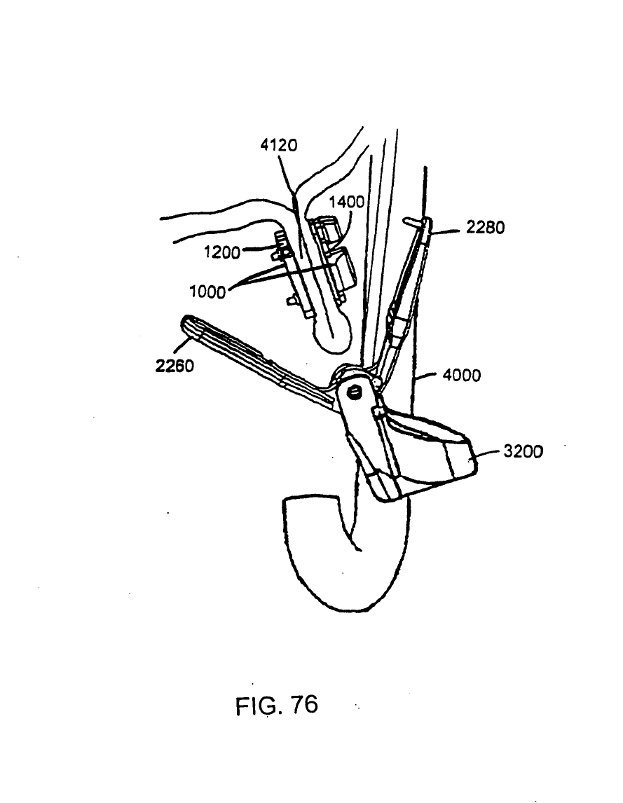

Referring to FIG. 76, if the plication 4120 appears satisfactory, the proximal actuation handle is operated to lock the male and female parts 1200, 1400 of the fastener 1000 and release the coupled fastener 1000 from the jaws 2260, 2280. If the plication or fastener position is not satisfactory, prior to locking and release, the jaws 2260, 2280 can be opened, reoriented if necessary, and another plication 4120 can be formed.

After the fastener 1000 is applied, the jaws 2260, 2280 are, then, closed, the endoscope 4000 is straightened, and the end effector 2020 is, if desired, re-docked over the distal end of the endoscope 4000. The clip implantation instrument and the endoscope 4000 are, together, withdrawn through the esophagus 4140 and out of the patient. Alternatively, the endoscope 4000 may be withdrawn first, followed by the withdrawal of the clip implantation instrument, preferably, under visualization with the endoscope.

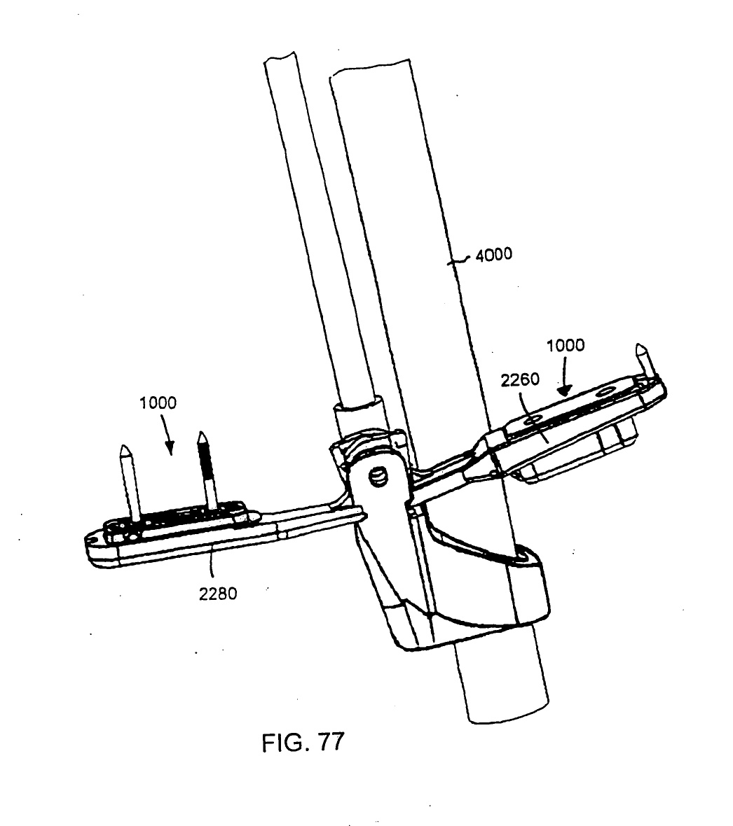

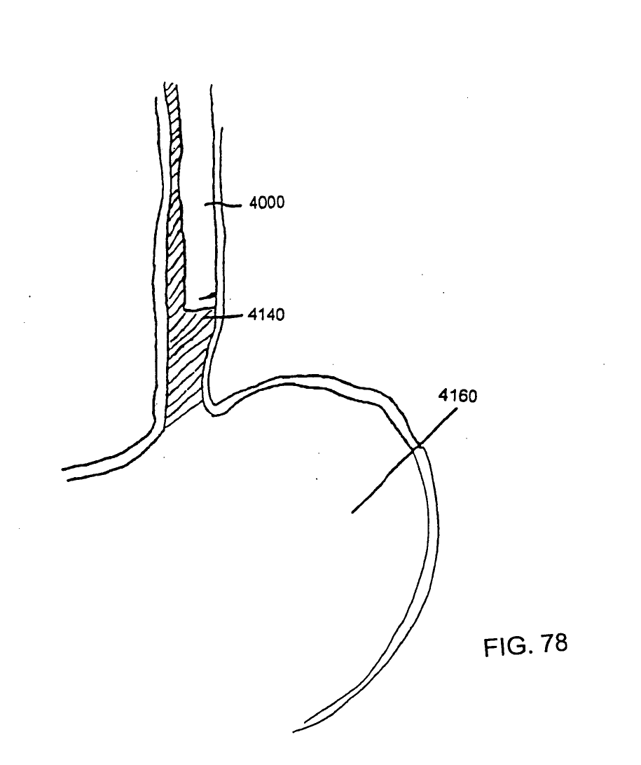

While it is preferable to decouple the clip implantation instrument from the endoscope 4000 during the procedure, it is appreciated that the clip implantation instrument may be operated while coupled to the endoscope. That is, referring to FIG. 77, the target tissue is approached by opening the jaws 2260, 2280 and simply retracting the end effector 2020 along the endoscope 4000 until the tissue 4100 about the LES 4020 is contacted. The jaws 2260, 2280 are, then, closed and the fastener 1000 applied, as described above. To utilize this procedure, the sleeve 3200 of the clip implantation instrument should be offset relative to the jaws 2260, 2280 so that the jaws 2260, 2280 can clear the endoscope 4000 when opening and closing.

While the clip implantation instrument has been shown adapted to be coupled to an endoscope, it is recognized that the clip implantation instrument may be modified for use in a manner in which it is always decoupled from an endoscope 4000.

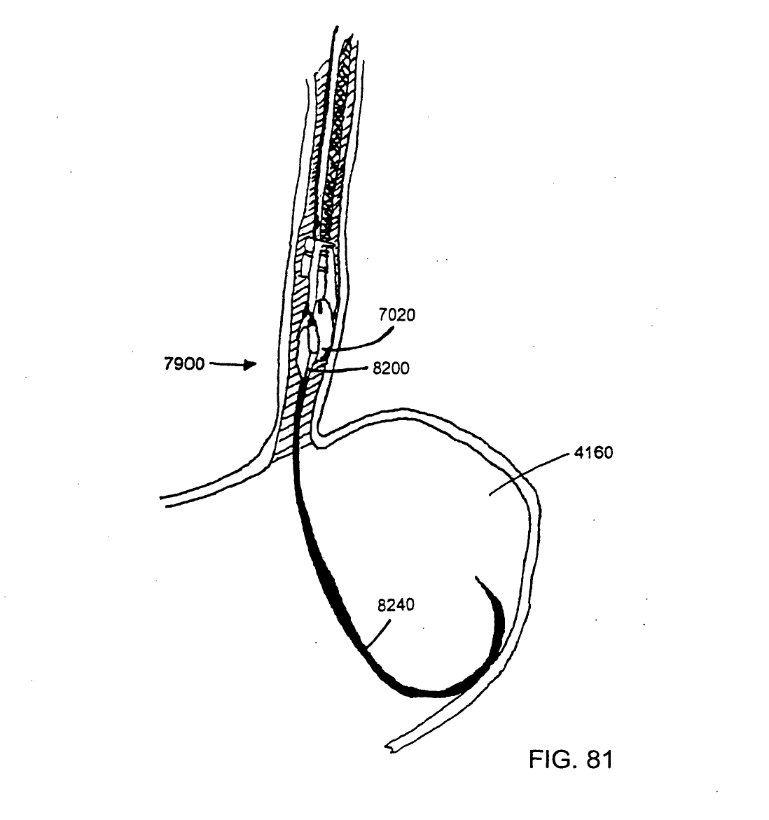

Referring now to FIGS. 81 to 88, a second alternate embodiment of the distal end effector 7020 of the clip implantation instrument is shown.

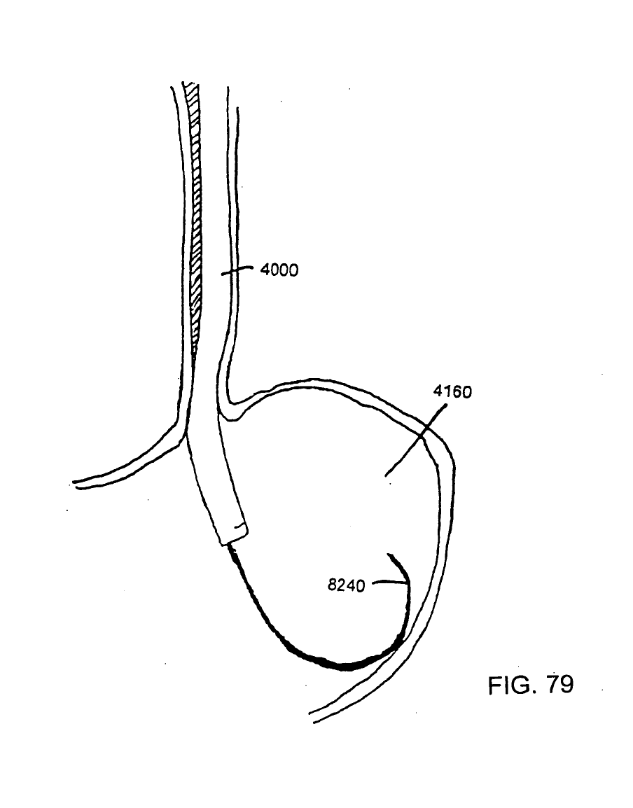

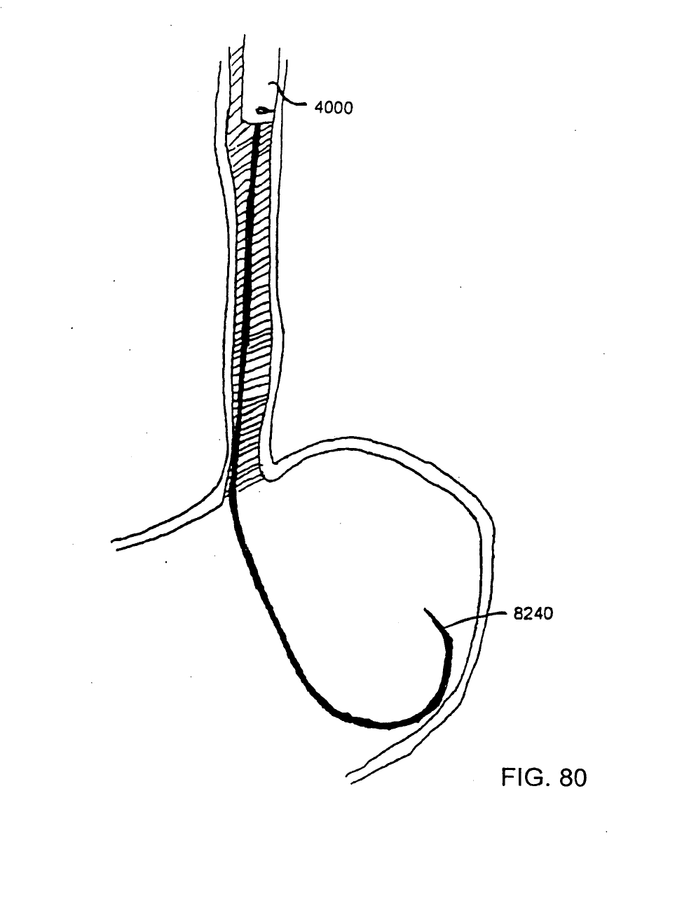

According to a preferred method of use, referring to FIG. 78, an endoscope 4000 is, preferably, first inserted through the tracheopharangeal passage 4140 and into the stomach 4160 in accord with a well-known procedure. Next, referring to FIG. 79, a guidewire 8240 is advanced through the endoscope into the stomach 4160. Referring to FIG. 80, the endoscope 4000 is, then, preferably withdrawn from over the guidewire 8240.

Referring to FIG. 81, the end effector 7020 is, then, blindly advanced over the guidewire 8240 and introduced into the stomach 4160. The housing 7900 of the end effector 7020 is provided with a tapered nosepiece 8200 defining a longitudinal passage 8220 sized to receive the guidewire 8240. The guidewire 8240 may have a diameter less than one millimeter, for example. Preferably, the nosepiece 8200 is formed from a highly flexible material such as silicone. The tapered nosepiece 8200 and relatively small head-on cross-sectional area of the system facilitate the introduction.

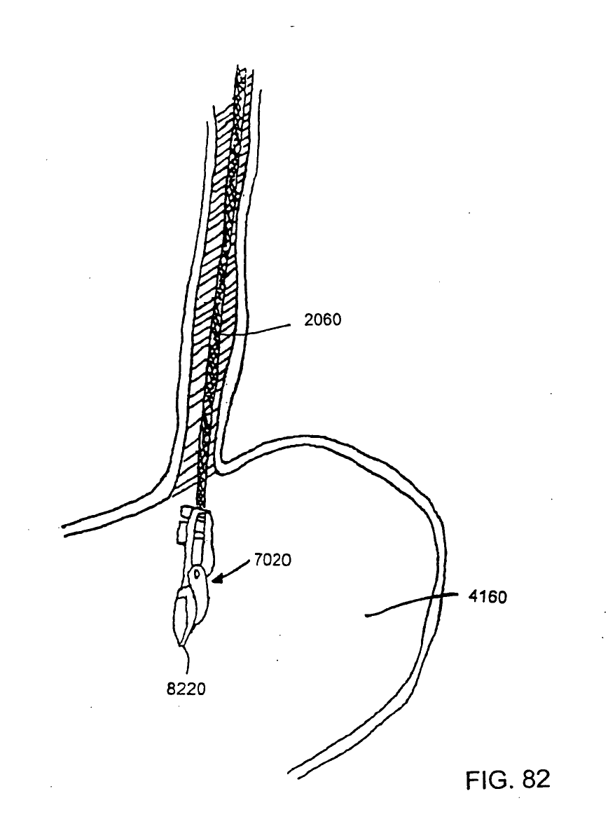

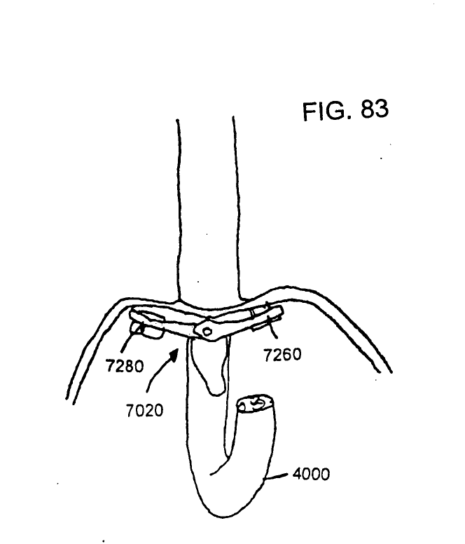

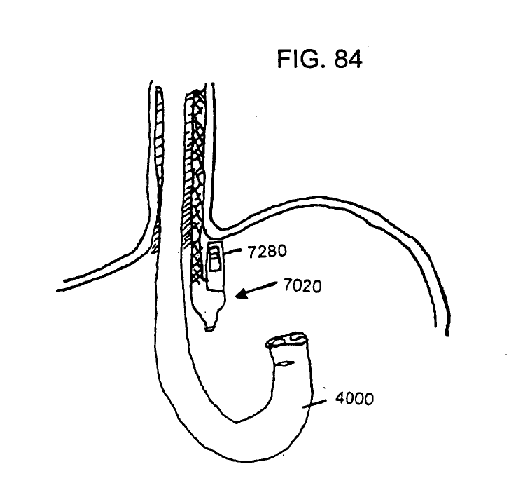

After the end effector 7020 is located in the stomach 4160, the guidewire 8240 is, preferably, withdrawn from the stomach 4160 as shown in FIG. 82. Referring now to FIGS. 83 and 84, the endoscope 4000 is, then, reintroduced alongside the control shaft 2060 of the clip implantation instrument, advanced into the stomach 4160, and retroflexed to view the end effector 7020. The jaws 7260, 7280 of the end effector 7020 are opened and brought adjacent the tissue that is to be plicated.

Referring to FIG. 85, the retractor 1 according to the present invention is deployed through a working channel 4080 of the endoscope 4000 and operated to engage tissue 9100 at a location at which the fold of a plication 4120 is desired. As set forth above, the needles 4 of the retractor 1 extend through the mucosa 202 and the muscularis 204 (deep muscle) to, thereby, hold these layers together and prevent delamination. A downward movement (with respect to FIG. 85) of the retractor 1 creates the desirable tent of stomach tissue 4100 mentioned above.

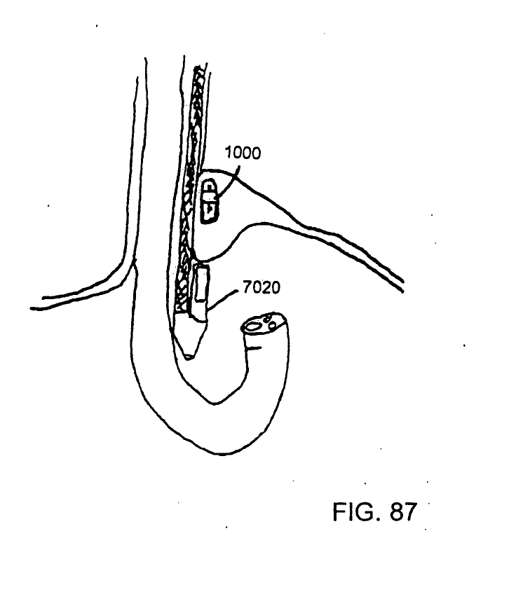

Turning to FIG. 86, the jaws of the end effector 7020 are closed about the tented tissue 4100, forming a plication 4120, the plication 4120 being substantially parallel to the esophagus 4140. The plication 4120 extends from the location held by the retractor 1 to the end of the jaws 7260, 7280 of the clip implantation instrument. Referring to FIG. 87, the fastener 1000 is deployed and the jaws 7260, 7280 of the end effector 7020 are opened.

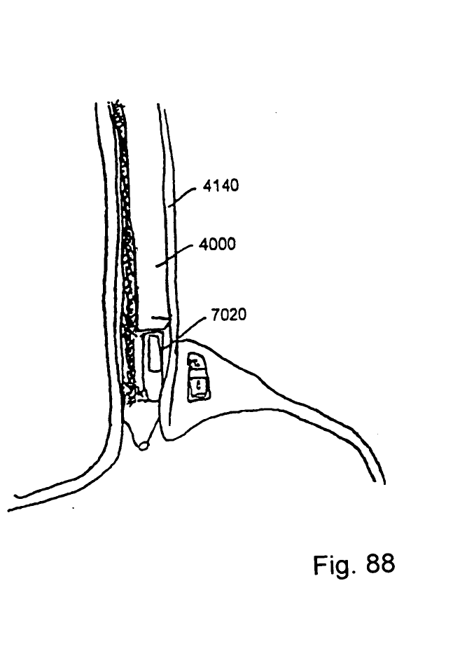

Finally, the jaws 7260, 7280 of the end effector 7020 are closed, as shown in FIG. 88, and the end effector 7020 is withdrawn through the esophagus 4140 under visualization of the endoscope 4000. That is, the closed jaws of the end effector 7020 are, preferably, positioned directly distal of the endoscope 4000 to minimize the cross-sectional area of the endoscope/clip implantation instrument system as well as to permit constant visualization of the end effector during the retraction of the end effector through the esophagus 4140.

Other embodiments of the invention will be apparent to those skilled in the art from consideration of the specification and practice of the invention disclosed herein. It is intended that the specification and examples be considered as exemplary only.

While the preferred embodiments of the invention have been illustrated and described, it will be clear that the invention is not so limited. Numerous modifications, changes, variations, substitutions, and equivalents will occur to those skilled in the art without departing from the spirit and scope of the present invention as defined by the appended claims.

There have been described and illustrated herein several embodiments of retractors and methods for the endoluminal treatment of Gastroesophageal Reflux Disease (GERD). While particular embodiments of the invention have been described, it is not intended that the invention be limited thereto, as it is intended that the invention be as broad in scope as the art will allow and that the specification be read likewise. For example, while particular preferred dimensions have been provided for the retractor, it is appreciated that the system and its elements may have different relative sizes. For example, the cross-sectional areas can be decreased further if a pediatric endoscope (4 to 6 mm) is used. Also, while a “looking back” clip implantation instrument has been disclosed particularly for fastener application designed to treat GERD, it is appreciated that a “forward looking” straight instrument with similar jaw assembly can be used to apply the fastener for treatments of other conditions, e.g., obesity, ulceration, stomach cancer, implantation of pH measurement or monitoring devices, feeding tubes, etc. Moreover, a straight device can be smaller in diameter and be operated through a working channel of an endoscope. It will, therefore, be appreciated by those skilled in the art that yet other modifications could be made to the provided invention without deviating from its spirit and scope as so claimed.

We claim:

1. A retractor for manipulating an object, comprising:
a body having proximal and distal ends;
a retraction device having:
a head connected to said distal end of said body, said head having a pivot;
substantially rigid needles pivotally connected to said head about said pivot; and
an actuator operatively connected to said needles and movably disposed within at least one of said head and said body; and
an actuation device connected to said proximal end of said body, said actuation device operatively connected to said actuator through said body and, upon actuation thereof, moving said actuator to selectively rotate said needles between a stowed position where said needles are stowed within said head and a fully extended position where said needles are extended out of said head.
